(19) United States
(12) Patent Application Publication
(10) Pub. No.: US 2024/0189101 A1
(43) Pub. Date: Jun. 13, 2024

(54) DOCKING STATIONS FOR PROSTHETIC IMPLANTS

(71) Applicant: EDWARDS LIFESCIENCES CORPORATION, Irvine, CA (US)

(72) Inventors: Alison Louise Rodriguez, Irvine, CA (US); Jensen Chen, Walnut, CA (US); Andrew Paul Schaffer, Orange, CA (US); Abdorruhman M. Yousef, Santa Ana, CA (US); John J. Desrosiers, San Clemente, CA (US); Rachel Christine Leacock, Torrance, CA (US); Anthony Michael Romero, Corona, CA (US)

(21) Appl. No.: 18/582,928

(22) Filed: Feb. 21, 2024

Related U.S. Application Data
(63) Continuation of application No. PCT/US2022/041991, filed on Aug. 30, 2022.
(60) Provisional application No. 63/239,334, filed on Aug. 31, 2021.

(51) Int. Cl. A61F 2/24; A61F 2/00; A61F 2/07

(52) U.S. Cl. CPC A61F 2/2418; A61F 2002/0081; A61F 2002/075; A61F 2210/0076; A61F 2250/0063; A61F 2250/0069

(57) ABSTRACT

A docking station for a prosthetic implant includes a frame. The frame includes one or more tissue engaging elements, which is some examples are apices of the frame formed by struts of the frame. The frame and/or the apices can be configured to engage native tissue at an implantation location to retain the position of the frame without damaging the native tissue. A docking station can also include a sealing skirt and/or a protective cover coupled to the frame. The sealing skirt can reduce retrograde blood flow through and/or around the frame. The protective cover can reduce damage to the native tissue.

### CROSS-REFERENCE TO RELATED APPLICATIONS

This application is a continuation of International Patent Application No. PCT/US2022/041991, filed Aug. 30, 2022, which claims the benefit of U.S. Provisional Patent Application No. 63/239,334, filed on Aug. 31, 2021. The prior applications are incorporated by reference herein.

### FIELD

The present disclosure relates generally to implantable prosthetic devices and more particularly to docking stations for prosthetic heart valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally invasive surgical approaches are used in various procedures to deliver prosthetic medical devices to locations inside the body that are not readily accessible by surgery or where access without surgery is desirable.

In one specific example, a prosthetic valve can be mounted in a crimped state on the distal end of a delivery apparatus and advanced through the patient's vasculature (e.g., through a femoral artery and the aorta) until the prosthetic valve reaches the implantation location in the heart. The prosthetic valve is then expanded to its functional size, for example, by inflating a balloon on which the prosthetic valve is mounted, actuating a mechanical actuator that applies an expansion force to the prosthetic valve, or by deploying the prosthetic valve from a sheath of the delivery apparatus so that the prosthetic valve can self-expand to its functional size.

In some cases, it may not be possible to secure the prosthetic valve to the native valve annulus, for example, if the native valve annulus is too large or if the geometry of the native valve is too complex to allow secure implantation of the valve. One approach in these cases is to first deploy a docking station at the implantation location and then install the prosthetic valve in the docking station. The docking station can be selected to provide the necessary interface to anchor the prosthetic valve within the native valve annulus. Desirably, the docking station can be delivered to the implantation location with a minimally invasive procedure, which would allow the docking station to be deployed within the same procedure used to deliver the prosthetic valve.

### SUMMARY

Disclosed herein are examples of prosthetic implants, such as docking stations, which can be implanted within a patient's body. The disclosed docking stations can, for example, be positioned within or adjacent a native heart valve annulus and be configured to receive a prosthetic heart valve. In this manner, the docking stations act as a support structure or anchor to help retain the positioning of the prosthetic heart valve relative to the native anatomy. The disclosed docking stations can comprise a frame having a plurality of struts. The frame can be radially compressible to a delivery configuration and radially expandable from the delivery configuration to a functional configuration. In some implementations, the frame can include one or more features configured to help retain the docking station relative to the native anatomy. For example, the frame can comprise a contoured shape corresponding to the shape of the native anatomy. Additionally or alternatively, the frame can comprise one or more apices formed by the struts and configured to engage the native tissue. In particular examples, the apices and/or other portions of the frame can comprise a cover coupled thereto. The cover can, for instance, help prevent or reduce the likelihood that the apices will damage (e.g., puncture and/or tear) the native tissue.

A docking station can include a frame (which can also be called a “stent” or a “prestent”) comprising a plurality of struts. The struts can be interconnected in a manner that allows the struts to move between a radially-compressed state and a radially-expanded state.

In some examples, a docking station for a prosthetic implant includes a frame and one or more protective covers. The frame includes a plurality of struts, and the struts form one or more apices. The protective covers are disposed on the apices and are configured to be positioned between the apices of the frame and native tissue at an implantation location.

In some examples, a frame for supporting a prosthetic implant, including a first plurality of cells and a second plurality of cells. The first plurality of cells is arranged in a first circumferentially-extending row. The second plurality of cells is arranged in a second circumferentially-extending row, and the cells of the second plurality of cells are larger than the cells of the first plurality of cells.

In some examples, a sealing skirt for a docking station includes a first portion and a second portion. The first portion is configured to cover one or more cells of the frame, and the second portion is configured to extend between adjacent cells of the frame.

In some examples, a frame for a docking station includes a plurality of cells and one or more support struts. The plurality of cells is defined by a plurality of struts, and the cells comprise a first row of apices and a second row of apices. Each support strut extends axially from an apex in the first row of apices to an apex in the second row of apices.

In some examples, a frame for a docking station includes a plurality of struts forming a plurality of cells. The cells extend from an inflow end of the frame to an outflow end of the frame. One or more cells disposed adjacent the outflow end comprise a radially tapered section, and one or more cells disposed adjacent the inflow end comprise a radially curved section.

In some examples, a frame for a docking station includes an inflow end portion, an outflow end portion, and an intermediate portion. The outflow end portion has a first diameter at a first axial location and a second diameter at a second axial location. The second diameter is smaller than the first diameter. The second axial location is disposed closer to a distal end of the frame than the first axial location. The intermediate portion is disposed between the inflow end portion and the outflow end portion and having a third diameter at a third axial location. The third diameter is smaller than the first diameter and the second diameter. The third axial location is disposed closer toward an inflow end of the frame than the first axial location and the second axial location.

The above devices can be used as part of an implantation procedure performed on a living animal or on a simulation, such as on a cadaver, cadaver heart, anthropomorphic ghost, simulator (e.g., with body parts, heart, tissue, etc. being simulated).

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is an elevation view of a portion of a frame of a docking station in a radially-expanded state.

FIG. 2 is a perspective view of the frame of FIG. 1 in a radially-compressed state.

FIG. 3 is a perspective view of a docking station including the frame of FIG. 1.

FIG. 4 is a cut-away view of the docking station of FIG. 3 deployed at an implantation location within a patient's anatomy, which is depicted schematically in cross-section, and with a prosthetic heart valve deployed therein.

FIG. 5A is a perspective view of a delivery apparatus for deploying a docking station.

FIG. 5B illustrates the docking station of FIG. 3 disposed around a distal portion of the delivery apparatus of FIG. 5A.

FIG. 6A is an elevation view of a distal portion of the delivery apparatus of FIG. 5A with an outer shaft of the delivery apparatus in a retracted position.

FIG. 6B is an elevation view of a distal portion of the delivery apparatus of FIG. 5A with an outer shaft of the delivery apparatus in an extended position and cut away to show an encapsulated docking station.

FIGS. 6C-6F illustrate stages in deployment of the docking station of FIG. 3 from the delivery apparatus of FIG. 5A.

FIG. 7A is a perspective view of a handle portion of the delivery apparatus illustrated in FIG. 5A.

FIGS. 7B and 7C are perspective views of the handle portion of FIG. 7A with a portion of the handle cut away to show various internal components.

FIG. 8 is a perspective view of an exemplary docking station.

FIG. 9 is a perspective view of an inflow end portion of a frame of the docking station of FIG. 8.

FIG. 10 is a side view of a portion of a sealing skirt having a protective cover portion, according to one example.

FIG. 11 is a side view of a portion of a sealing skirt having a protective cover portion, according to another example.

FIG. 12 is a side view of a portion of a sealing skirt having a protective cover portion, according to another example.

FIGS. 13-14 depict various views of an exemplary docking station partially deployed from the delivery apparatus of FIG. 5A.

FIG. 15 is a perspective view of another exemplary docking station.

FIG. 16 is a plan view of a portion of a sealing skirt of the docking station of FIG. 15.

FIG. 17 is a plan view of a portion of another exemplary sealing skirt for a docking station.

FIG. 18 is a side view of another exemplary docking station.

FIG. 19 is an outflow end view of the docking station of FIG. 18.

FIG. 20 is a perspective view of an exemplary frame for a docking station.

FIG. 21 is a perspective view of an exemplary frame for a docking station.

FIG. 22A is a perspective view of an exemplary docking station comprising the frame of FIG. 21, depicting connector tabs of the frame curved similar to inflow apices of the frame.

FIG. 22B is a perspective view of an exemplary docking station comprising the frame of FIG. 21, depicting connector tabs of the frame flared radially outwardly relative to inflow apices of the frame.

FIG. 23 is a side view of an exemplary frame for a docking station.

FIG. 24 is an outflow end view of the frame of FIG. 23.

FIG. 25 is a side view of an exemplary frame for a docking station.

FIG. 26A is a schematic view of a tapered end portion the frame of FIG. 25.

FIG. 26B is a schematic view of a curved end portion of a frame for purposes of comparison to the frame depicted in FIGS. 25 and 26A.

FIG. 27 is perspective view of the frame of FIG. 25 and an exemplary sealing skirt.

FIG. 28 is perspective view of the frame of FIG. 25 and another exemplary sealing skirt.

FIG. 29 is a side view of an exemplary frame for a docking station.

FIG. 30 is an outflow end view of the frame of FIG. 29.

FIG. 31 is a perspective view of another exemplary docking station.

FIG. 32 is a table depicting an exemplary configuration for the apices of the frame of the docking station depicted in FIG. 31.

FIG. 33 depicts an end portion of an exemplary frame for a docking station.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

In the interest of conciseness, and for the sake of continuity in the description, same or similar reference characters may be used for same or similar elements in different figures, and description of an element in one figure will be deemed to carry over when the element appears in other figures with the same or similar reference character. In some cases, the term “corresponding to” may be used to describe correspondence between elements of different figures. In an example usage, when an element in a first figure is described as corresponding to another element in a second figure, the element in the first figure is deemed to have the characteristics of the other element in the second figure, and vice versa, unless stated otherwise.

As used in this application and in the claims, the singular forms “a,” “an,” and “the” include the plural forms unless the context clearly dictates otherwise. The word “comprise” and derivatives thereof, such as “comprises” and “comprising,” are to be construed in an open, inclusive sense, that is, as “including, but not limited to.” Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while distal motion of the device is motion of the device away from the user and toward the implantation site (e.g., into the patient's body). The terms “longitudinal” and “axial” refer to an axis extending in the proximal and distal directions, unless otherwise expressly defined.

As used herein, the term “simulation” means a performing an act on a cadaver, cadaver heart, anthropomorphic ghost, and/or a computer simulator (e.g., with the body parts, tissue, etc. being simulated).

### Introduction to the Disclosed Technology

As mentioned above, the docking stations disclosed herein can comprise a frame having a plurality of struts. The struts of the frame can, in some instances, form one or more apices at the inflow and/or outflow ends of the frame. In some implementations, the frame can include one or more features configured to help retain the docking station relative to the native anatomy. For example, the frame can comprise a contoured shape (e.g., an hourglass shape) corresponding to the shape of the native anatomy. Additionally or alternatively, the apices of the frame can engage the native tissue to help prevent or reduce migration of the docking station relative to the native tissue.

Although engagement between the frame (e.g., the apices) and the native tissue can be beneficial for preventing migration of the docking station relative to the native tissue, it can also be beneficial to control the extent to which the frame engages the native tissue. For example, limiting or reducing engagement between the frame and the native tissue can, for instance, help prevent the frame from puncturing, tearing, and/or otherwise damaging the native tissue.

Thus, disclosed herein are various frame configurations (e.g., shapes) and/or other features configured to allow the docking station to have sufficient anti-migration relative to the native anatomy, while also reducing the likelihood of tissue damage. For example, the docking stations disclosed herein can comprise one or more covers and/or frame shapes configured to prevent or reduce the likelihood that the frame (e.g., the apices) will damage the native tissue.

### Examples of the Disclosed Technology

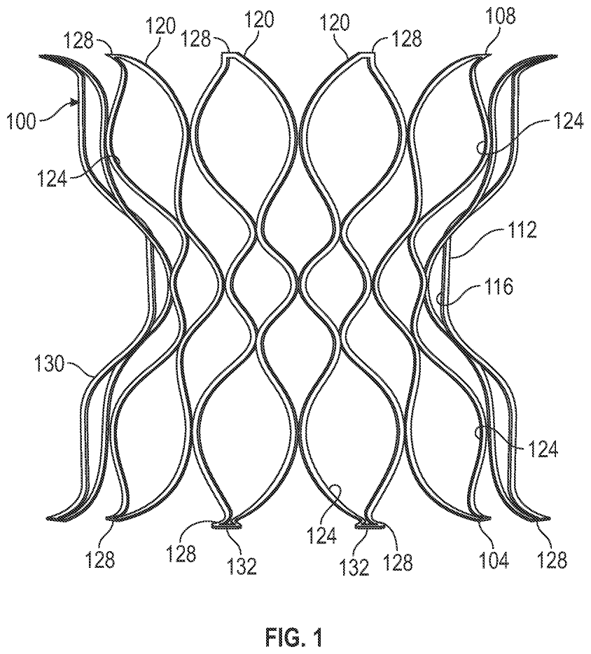

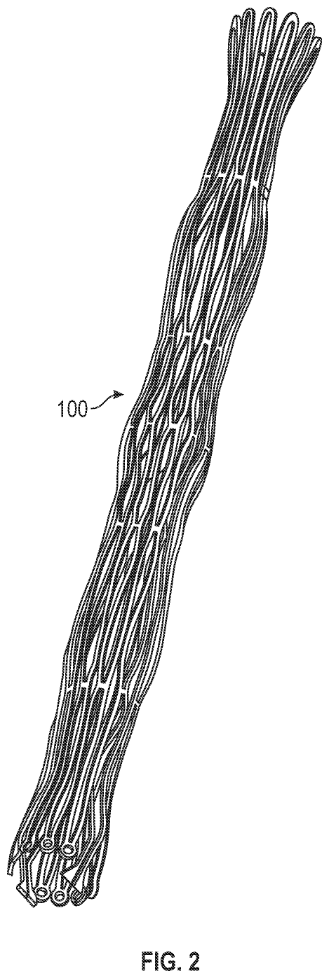

Turning now to the drawings, FIG. 1 illustrates an exemplary implementation of a frame 100 (or stent) that can form a body of a docking station. The frame 100 has a first end 104 and a second end 108. In some examples, the first end 104 can be an inflow end, and the second end 108 can be an outflow end. In some examples, the first end 104 can be an outflow end, and the second end 108 can be an inflow end. The terms “inflow” and “outflow” are related to the normal direction of blood flow (e.g., antegrade blood flow) through the frame. In the unconstrained, expanded state of the frame 100 shown in FIG. 1, a relatively narrower portion (or waist) 112 of the frame 100 between the first end 104 and the second end 108 forms a valve seat 116. The frame 100 can be compressed (as illustrated in FIG. 2) for delivery to an implantation location by a delivery apparatus.

Although the docking stations, delivery apparatus, prosthetic heart valves, and/or methods are described herein with respect to a particular implantation location (e.g., a pulmonary valve) and/or a particular delivery approach (e.g., transfemoral), the device and methods disclosed herein can be adapted to various other implantation locations (e.g., an aortic valve, a mitral valve, and a tricuspid valve) and/or delivery approaches (e.g., transapical, transseptal, etc.).

In the example illustrated by FIG. 1, the frame 100 includes a plurality of struts 120 arranged to form cells 124. The ends of the struts 120 form apices 128 at the ends of the frame 100. One or more of the apices 128 can include a connector tab 132. The portions of the struts 120 between the apices 128 and the valve seat 116 (or the waist 112) form a sealing portion 130 of the frame 100. In the unconstrained, expanded state of the frame 100 illustrated in FIG. 1, the apices 128 extend generally radially outward and are radially outward of the valve seat 116.

The frame 100 can be made of a highly resilient or compliant material to accommodate large variations in the anatomy. For example, the frame 100 can be made of a flexible metal, metal alloy, polymer, or an open cell foam. An example of a highly resilient metal is nitinol, which is a metal alloy of nickel and titanium, but other metals and high resilient or compliant non-metal materials can be used. The frame 100 can be self-expanding, manually expandable (e.g., expandable via a balloon), or mechanically expandable. A self-expanding frame can be made of a shape memory material, such as, for example, nitinol. In this manner, the frame can be radially compressed as depicted in FIG. 2 (e.g., via a crimping device) and can radially expand to the configuration depicted in FIG. 1.

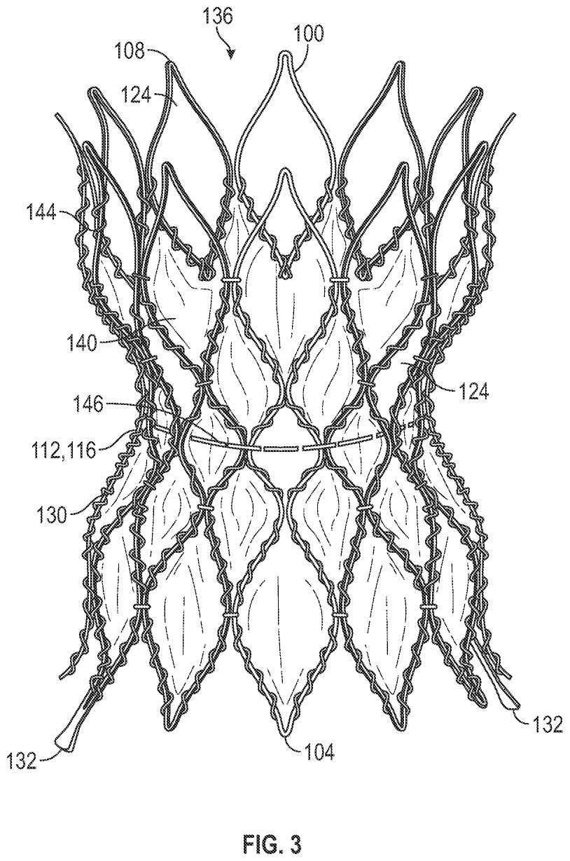

FIG. 3 illustrates an exemplary docking station 136 including the frame 100 and a sealing skirt 140 disposed within the frame. The sealing skirt 140 is attached to the frame 100 (e.g., by sutures 144). In the example illustrated by FIG. 3, the sealing skirt 140 covers at least the cells 124 in the sealing portion 130 of the frame 100. In this manner, the sealing skirt 140 can help funnel blood flowing into the docking station 136 from the proximal inflow end 104 to the valve seat 116 (and the valve once installed in the valve seat). The sealing skirt can additionally or alternatively help to prevent or reduce parastent leakage (e.g., retrograde blood flow between the docking station and the native tissue and/or paravalvular leakage (e.g., retrograde blood flow between the prosthetic heart valve and the docking station. In the illustrated example, the row of cells proximate to the distal outflow end 108 is not covered by the sealing skirt 140. The uncovered cells can, for example, permit blood to flow through the distal side of the frame and/or enable catheter access to the left and right pulmonary arterial branches (e.g., to pass another intravascular device (e.g., catheter, wire, etc.) during the implantation procedure and/or during a secondary intervention).

The sealing skirt 140 can be a fabric that is impermeable to blood. A variety of biocompatible materials can be used for the sealing skirt 140, such as, for example, foam or a fabric that is treated with a coating that is impermeable to blood, a polyester material, or a processed biological material, such as pericardium. In some examples, the sealing skirt 140 can comprise polymeric material, including polyethylene terephthalate (PET), expanded polytetrafluorethylene (ePTFE), and/or thermoplastic polyurethane (TPU).

The docking station 136 may include a band 146 that extends around the waist 112 (or that is integral to the waist) of the frame 100. The band 146 can constrain expansion of the valve seat 116 to a specific diameter in the deployed state to enable the valve seat 116 to support a specific valve size. The band 146 can take on a wide variety of different forms and can be made of a wide variety of different materials. For example, the band 146 can be made of PET, one or more sutures, fabric, metal, polymer, a biocompatible tape, or other relatively nonexpanding materials known in the art and that can maintain the shape of the valve seat 116.

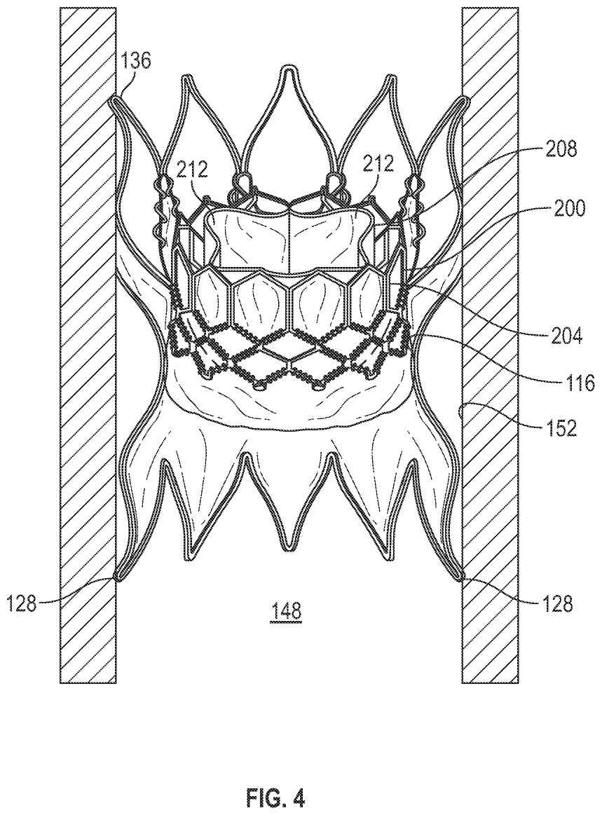

FIG. 4 illustrates the docking station 136 in a deployed state within a native valve annulus 148 (shown schematically). As can be seen, the frame 100 of the docking station 136 is in an expanded condition, with the end portions of the frame pressed against the inner surface 152 of the native valve annulus. The band 146 (shown in FIG. 3) can maintain the valve seat 116 at a constant or substantially constant diameter in the expanded condition of the frame 100. FIG. 4 also shows a prosthetic valve 200 deployed within the docking station 136 and engaged with the valve seat 116 of the docking station 136. The prosthetic valve 200 can be implanted by first deploying the docking station 136 at the implantation location and then installing the prosthetic valve within the docking station.

The prosthetic valve 200 can be configured to replace a native heart valve (e.g., aortic, mitral, pulmonary, and/or tricuspid valves). In one example, the prosthetic valve 200 can include a frame 204 and a valvular structure 208 disposed within and attached to the frame 204. The valvular structure 208 can include one or more leaflets 212 that cycle between open and closed states during the diastolic and systolic phases of the heart. The frame 204 can be made of the frame materials described for the frame 100 of the docking station 136. The leaflets 212 can be made in whole or in part from pericardial tissue (e.g., bovine pericardial tissue), biocompatible synthetic materials, or various other suitable natural or synthetic materials known in the art.

The docking station 136 is not limited to use with the particular example of the prosthetic valve 200 illustrated in FIG. 4. For example, mechanically expandable prosthetic valves may be installed in the docking station 136. Exemplary mechanically expandable prosthetic valves are described in U.S. Pat. Nos. 10,603,165 and 10,806,573 and International Application Nos. PCT/US2019/056865 and PCT/US2020/040318, which are incorporated by reference herein. Additional information about docking stations and prosthetic valves can be found in U.S. Pat. No. 10,363,130, which is incorporated by reference herein.

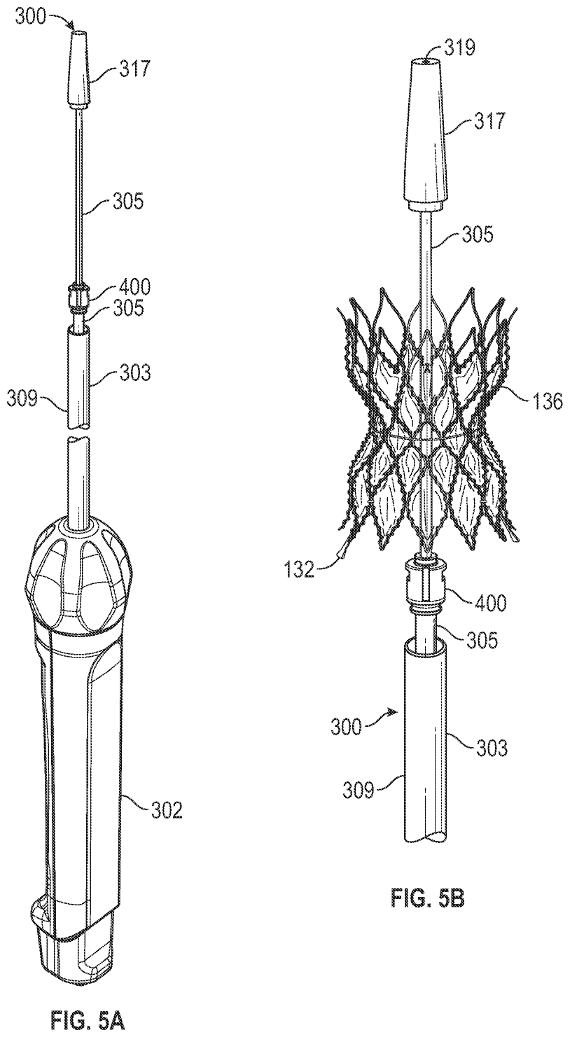

FIG. 5A illustrates an exemplary delivery apparatus 300 that can be used to deliver the docking station to an implantation location. The delivery apparatus 300 generally includes a handle 302 and a shaft assembly 303 coupled to the handle 302 and extending distally from the handle 302. The shaft assembly 303 includes an inner shaft 305 and an outer shaft 309. The inner shaft 305 extends through a lumen of the outer shaft 309.

In the example illustrated by FIG. 5A, a frame connector 400 is coupled to the inner shaft 305. The docking station 136 can be disposed around a portion of the inner shaft 305 extending distally from the frame connector 400, as shown in FIG. 5B. In one example, the frame connector 400 includes one or more recesses that can receive one or more connector tabs 132 at the proximal end of the docking station 136 and thereby axially restrain the docking station 136.

A nosecone 317 can be attached to a distal end of the inner shaft 305. The nosecone 317 includes a central opening 319 for receiving a guidewire. As such, a proximal end of the guidewire can be inserted into the central opening 319 and through the inner shaft 305, and a distal end portion of the delivery apparatus 300 can be advanced over the guidewire through a patient's vasculature and to an implantation location. The guidewire can pass through the nosecone 317 into the inner shaft 305 during advancing of the delivery apparatus through a patient's vasculature.

The handle 302 can be operated to move the outer shaft 309 relative to the inner shaft 305, generally between an extended position and a retracted position. The handle 302 can be extended to slide the outer shaft 309 over the frame connector 400 and over any docking station coupled to the frame connector 400 to encapsulate the docking station within the outer shaft 309. As the outer shaft 309 slides over the docking station 136, the outer shaft 309 can compress the docking station 136 such that the docking station is encapsulated within the outer shaft 309 in the compressed state. In the fully extended position, a distal end of the outer shaft 309 can abut a proximal end of the nosecone 317 such that there are no gaps in the delivery assembly. Additionally or alternatively, a crimping device can be used to radially compress the docking station such that it can be inserted into the outer shaft of the delivery apparatus.

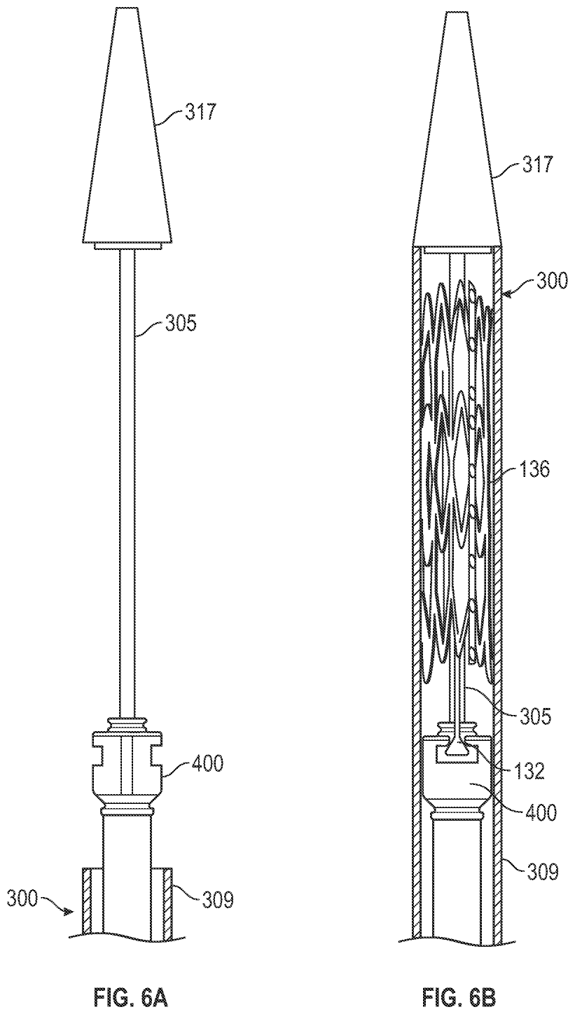

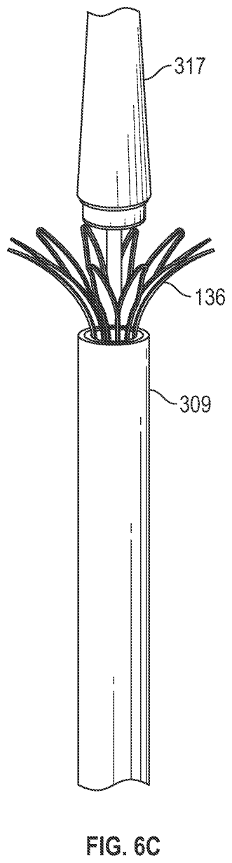

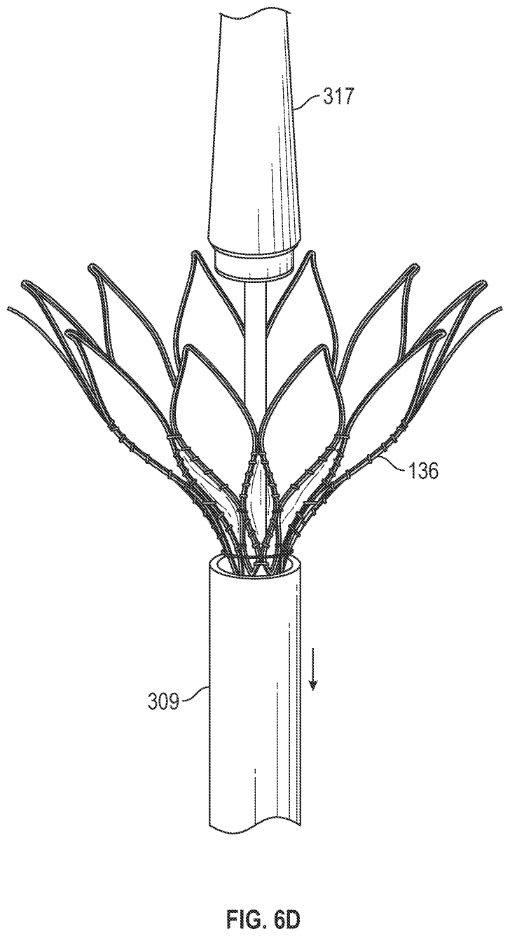

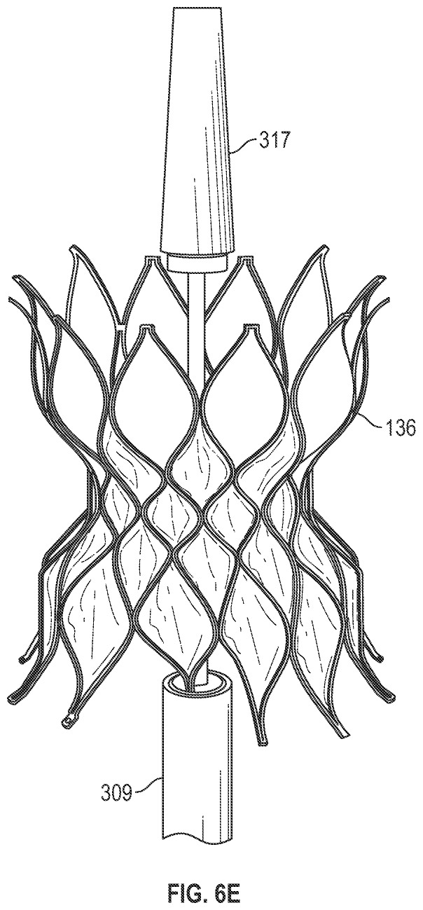

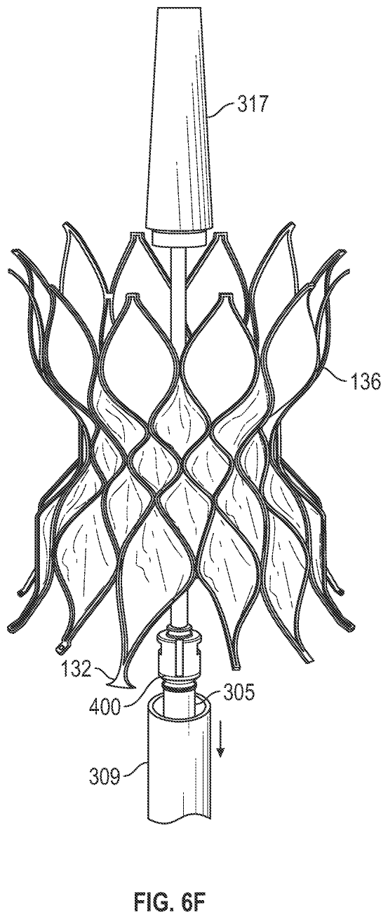

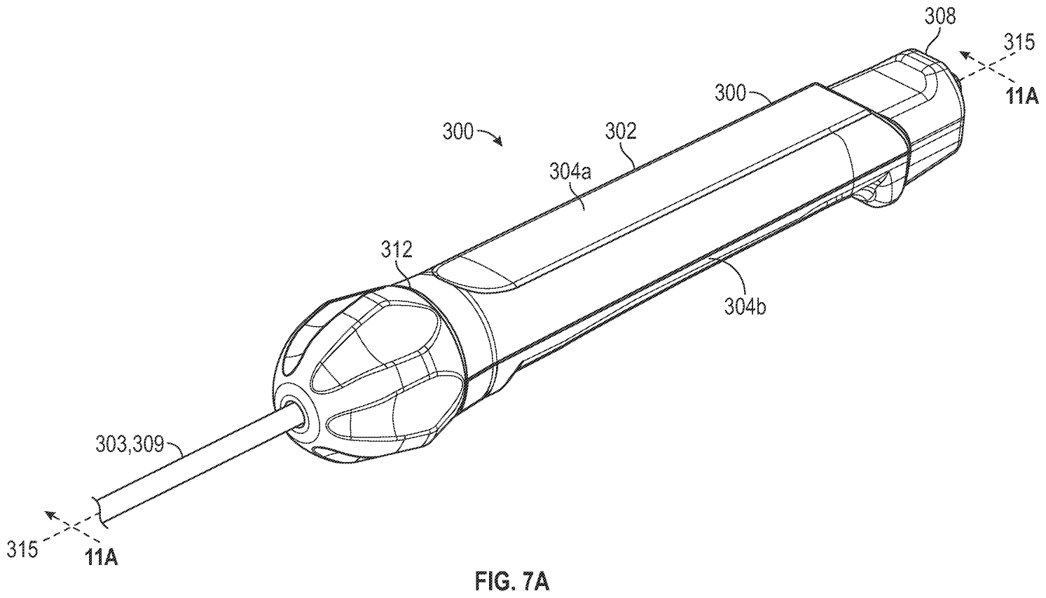

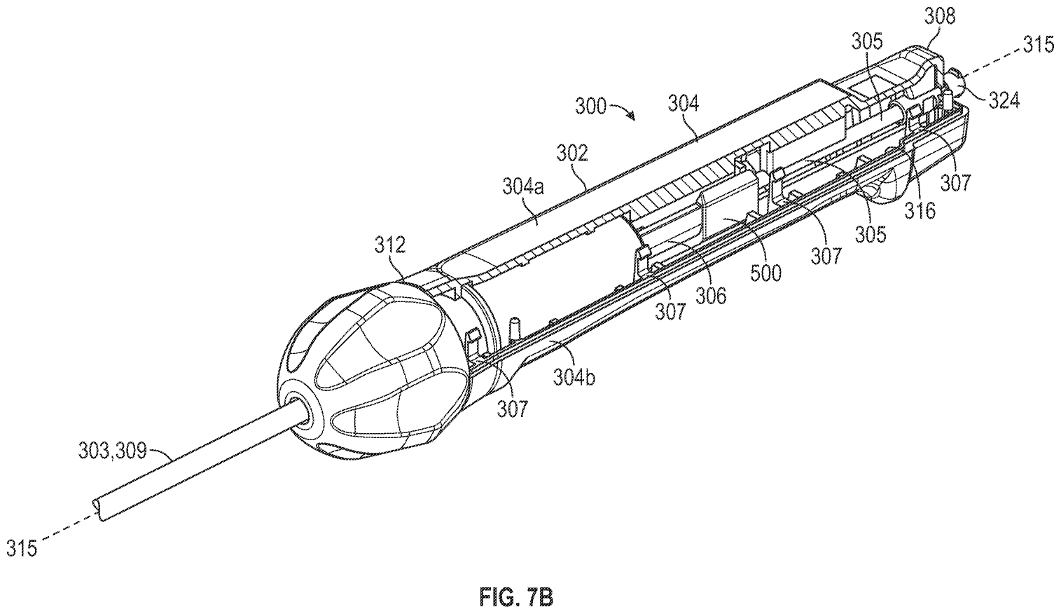

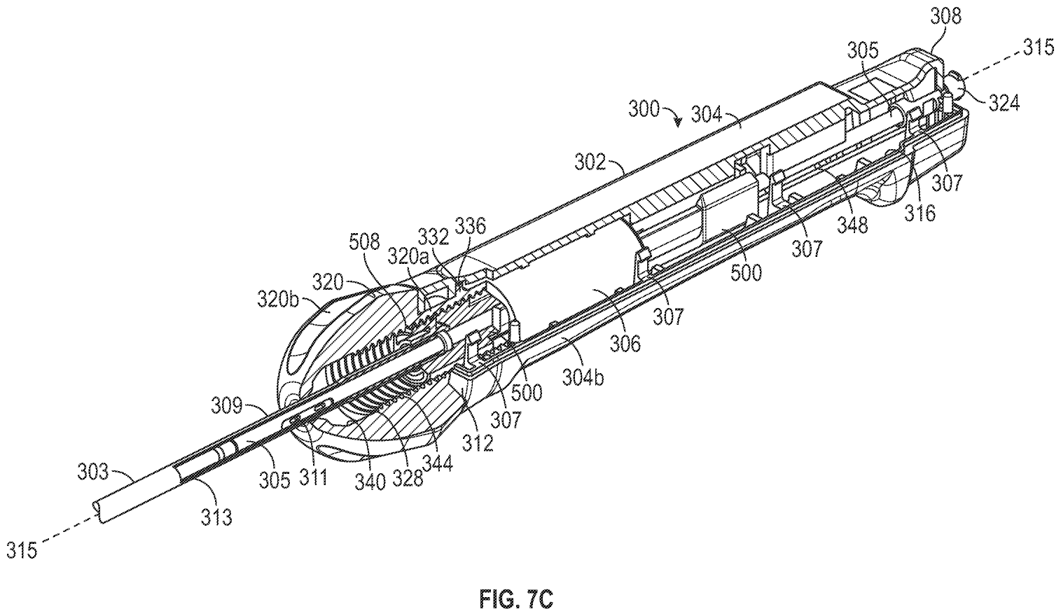

FIGS. 6A-7C illustrate a method of deploying a docking station at an implantation location within an anatomy. For purposes of illustration, the patient's anatomy is omitted. In FIG. 6A, the method includes retracting the outer shaft 309 by the handle of the delivery apparatus to allow loading of the docking station 136 onto the inner shaft 305. In FIG. 6B, the method includes disposing the docking station 136 around the inner shaft 305 and engaging each of the connector tabs 132 of the docking station 136 with the frame connector 400. The method also includes positioning the outer shaft 309 over the docking station such that the docking station is encapsulated therein. This can be accomplished by manipulating the handle of the delivery apparatus. As shown in FIG. 6B, the distal end of the outer shaft 309 abuts the proximal end of the nosecone 317. The method includes inserting the delivery apparatus, from the nosecone 317 end, into a patient's vasculature and advancing the delivery apparatus through the patient's vasculature to the implantation location.

At the implantation location, the method includes retracting the outer shaft 309 by the handle of the delivery apparatus to expose the docking station 136. FIGS. 6C-6F show different stages of retracting the outer shaft 309. As can be seen, in cases where the docking station 136 is self-expanding, the docking station 136 gradually emerges from the outer shaft 309 and gradually expands from the compressed state as the outer shaft 309 is retracted. When the outer shaft 309 is sufficiently retracted, the connector tabs 132 disengage from the frame connector 400. Once the docking station 136 is disengaged from the frame connector 400, the docking station 136 can radially expand to engage the anatomy.

FIGS. 7A-7C illustrate an exemplary implementation of the handle 302 of the delivery apparatus. The handle 302 includes a handle body 304 and a deployment mechanism 306 coupled to and partially disposed within the handle body. The handle body 304 includes a proximal end 308, a distal end 312, and a cavity 316 extending from the proximal end 308 to the distal end 312. The handle 302 includes a longitudinal axis 315 extending from the proximal end 308 to the distal end 312. The longitudinal axis 315 defines the axial direction of the handle.

The handle body 304 can be a single piece body with the cavity 316. Alternatively, the handle body 304 can have two body pieces 304a, 304b that can be assembled together to form the cavity 316. For example, the first body piece 304b may have snap hooks 307 that snap into complementary recesses in the second body piece 304a.

The deployment mechanism 306 of the handle 302 includes a carriage member 500 and a drive member 320. The carriage member 500 is disposed within the cavity 316 and movable relative to the handle body 304 in the axial direction. The drive member 320 engages with the carriage member 500 and is movable (e.g., rotatable) relative to the handle body 304 to adjust the axial position of the carriage member 500 relative to the handle body 304.

Proximal portions of the shafts 305, 309 are inserted into the cavity of the handle body 304. A proximal end portion of the outer shaft 309 of the shaft assembly 303 can be coupled to the carriage member 500 (e.g., by fasteners, adhesive, and/or other means for coupling) such that movement of the carriage member 500 relative to the handle body 304 causes movement of the outer shaft 309 between the extended and retracted positions.

A proximal portion of the inner shaft 305 extends through a lumen 313 of the outer shaft 309 into a proximal portion of the cavity 316 and is coupled to the handle body 304. The inner shaft 305 can be fixed relative to the handle body 304 such that the inner shaft 305 is stationary while the outer shaft 309 moves relative to the handle body 304.

In the example illustrated by FIGS. 7A-7C, an injection port 324 is mounted at an opening at the proximal end 308 of the handle body 304. The injection port 324 can be, for example, a Luer fitting. A proximal end of the inner shaft 305 can be inserted into the injector port 324 (shown in FIG. 11A) and secured to the injection port 324 (e.g., by bonding). In some cases, the attachment of the inner shaft 305 to the injection port 324 can serve the purpose of fixing the inner shaft 305 relative to the handle body 304.

The injection port 324 can be used to inject flushing fluid, such as saline, into the lumen of the inner shaft 305. In some cases, the inner shaft 305 can include one or more fluid ports 311 through which the injected fluid exits the inner shaft 305 and enters the lumen 313 of the outer shaft 309, thereby allowing flushing of the lumens of the inner shaft 305 and outer shaft 309 from a single injection port.

Additional details regarding the delivery apparatus and its components can be found in U.S. Application Nos. 63/154,956 and 63/154,966 and International Application No. PCT/US2022/018093, which are incorporated by reference herein.

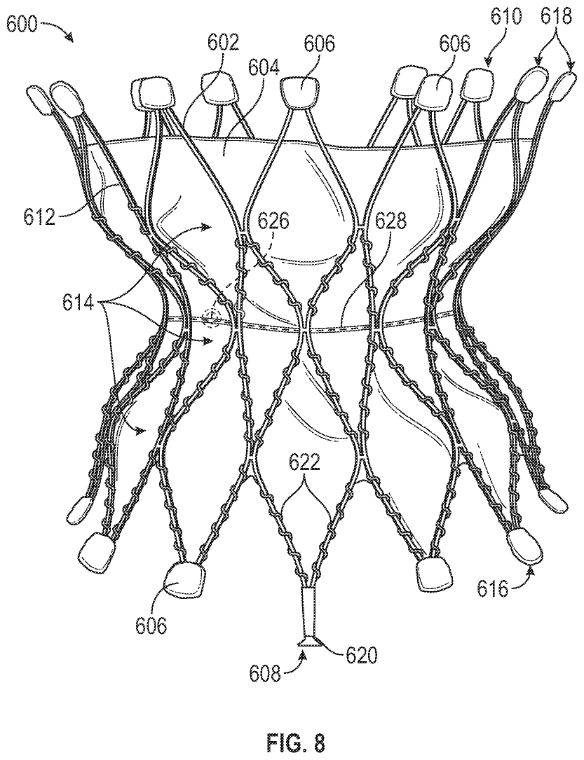

FIGS. 8-33 depict several additional examples of docking stations and/or their components. These docking stations comprise one or more features (e.g., covers and/or frame shapes) configured to configured to prevent or reduce the likelihood that the frame will damage the native tissue.

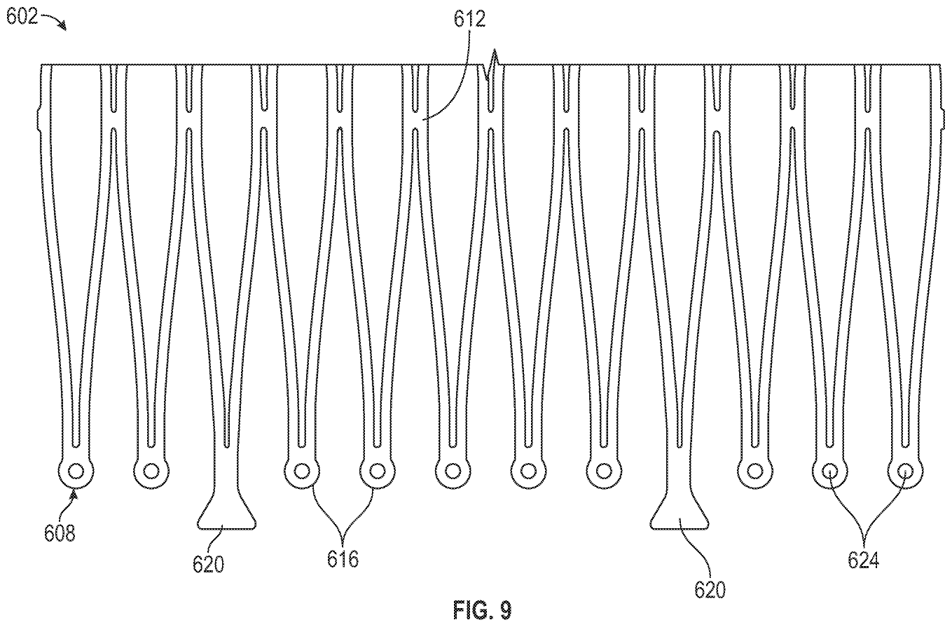

FIGS. 8-9 depict an example of a docking station 600 and its components. Referring to FIG. 8, the docking station 600 comprises three main components: a frame 602, a sealing skirt 604, and a plurality of protective covers 606. The frame 602 can be configured to engage native tissue at an implantation location (e.g., a native pulmonary valve) and configured to support a prosthetic heart valve therein. The sealing skirt 604 is coupled to the frame 602 can be configured to help reduce parastent and/or paravalvular leakage and/or to help promote tissue ingrowth. The protective covers are coupled to apices of the frame to prevent the native tissue from being damaged (e.g., punctured, torn, etc.). The docking station 600 also comprises an inflow end 608 and an outflow end 610.

With reference to FIGS. 8-9, the frame 602 of the docking station 600 can be

configured in a manner similar to the frame 100. For example, the frame 602 is formed of a plurality of struts 612. The struts 612 can define a plurality of cells 614 (FIG. 9), a plurality of inflow apices 616 disposed at the inflow end 608 of the frame 602, and a plurality of outflow apices 618 disposed at the outflow end 610 of the frame 602. It should be noted that the inflow apices 616 and the outflow apices 618 of the frame 602 are concealed by the protective covers 606 in FIG. 8. FIG. 9 depicts the inflow apices 616 of the frame 602 without the protective covers 606. The frame 602 can also comprise a plurality of connector tabs 620 configured for coupling the frame 602 to a delivery apparatus.

The cells 614 of the frame 602 are arranged in three circumferentially-extending rows. The shape/size of the cells, the number of cells, and/or the number of rows of cells can be altered from the illustrated example.

Referring again to FIG. 8, the sealing skirt 604 can, for instance, help reduce parastent and/or paravalvular leakage. The sealing skirt 604 of the docking station 600 extends from the inflow apices 616 of the frame 602 toward the outflow end 610 of the frame 602 but does not extend completely to the outflow apices 618. As such, a portion (e.g., 10-30% or 20-25%) of the cells of the frame adjacent the outflow end is unobstructed by the sealing skirt 604. The unobstructed portions of the cells can, for example, permit blood to flow through the distal side of the frame 602 and/or enable catheter (or other intravascular device) access to the left and right pulmonary arterial branches. The unobstructed portions of the cells can additionally or alternatively provide a path to pass another intravascular device (e.g., catheter, wire, etc.) during a subsequent intervention.

In other examples, the sealing skirt can extend from the inflow apices of the frame to the outflow apices of the frame and completely cover the cells of the frame. In some examples, at least a portion (e.g., 10-30% of 20-25%) of the cells adjacent the inflow end of the frame can be unobstructed by the sealing skirt.

In the illustrated example, the sealing skirt 604 is disposed on the interior surface of the frame and not on the exterior surface of the frame. In some examples, the sealing skirt can be disposed on the exterior surface of the frame and not on the interior surface of the frame. In some examples, the sealing skirt can be disposed on one or more portions of the interior surface of the frame and one or more portions of the exterior surface of the frame. In some examples, a plurality of sealing skirts can be provided, such as a first sealing skirt disposed on the interior surface of the frame and a second sealing skirt disposed on the exterior surface of the frame.

The sealing skirt 604 is a single, continuous piece of material. In some examples, the sealing skirt can comprise a plurality of sections that are coupled together (e.g., via sutures).

The sealing skirt 604 can be coupled to the frame 602 in various ways. For example, the sealing skirt 604 can be coupled to the frame with fasteners (e.g., clips), sutures, adhesive, and/or other means for coupling. In the illustrated example, the sealing skirt 604 is coupled to the frame 602 with sutures 622.

The sealing skirt 604 can be formed of various materials, including cloth. The cloth can be woven or non-woven materials. In some examples, the sealing skirt can comprise PET, PTFE, ePTFE, TPU, and/or other materials.

Referring still to FIG. 8, the protective covers 606 of the docking station 600 are disposed over the inflow apices 616 and the outflow apices 618 of the frame 602. As mentioned above, the protective covers 606 can, for example, prevent or reduce the likelihood of the native tissue from being damaged (e.g., punctured, torn, etc.). The protective covers 606 can also (or alternatively) promote tissue ingrowth.

The protective covers 606 can be formed of various materials, including cloth. The cloth can be woven or non-woven materials. In some examples, the protective covers 606 can comprise PET, PTFE, ePTFE, TPU, and/or other materials. In some instances, high-density materials (e.g., HD PET) are preferred over low-density materials to cushion frame edges without adding excessive bulk. Bulkier materials can, in some circumstances, promote excessive tissue ingrowth, which is not preferred. In configurations comprising woven materials, the cloth warp and weft can be oriented at a wide range of different angles with respect to the frame.

The protective covers 606 of the docking station 600 cover both the interior surfaces and the exterior surfaces of the apices of the frame. In some examples, the protective covers 606 of the docking station 600 cover the exterior surfaces of the apices of the frame, which are the surfaces that contact the native tissue.

The protective covers can be coupled to the apices of the frame in various ways, including fasteners (e.g., clips), sutures, adhesive, and/or other means for coupling. In the illustrated example, the protective covers 606 are coupled to the frame 602 and/or the sealing skirt 604 (e.g., at the inflow end) via sutures.

In some instances, the frame can comprise one or more attachment features (e.g., openings, recesses, projections, etc.) configured for attaching the protective covers to the apices. For example, as depicted in FIG. 9, the frame 602 comprises openings 624 formed in the inflow apices 616, which can be used for coupling the protective covers 606 to the apices (e.g., via sutures). Although not visible in FIG. 8, the outflow apices 618 comprise openings formed therein for coupling the protective covers thereto.

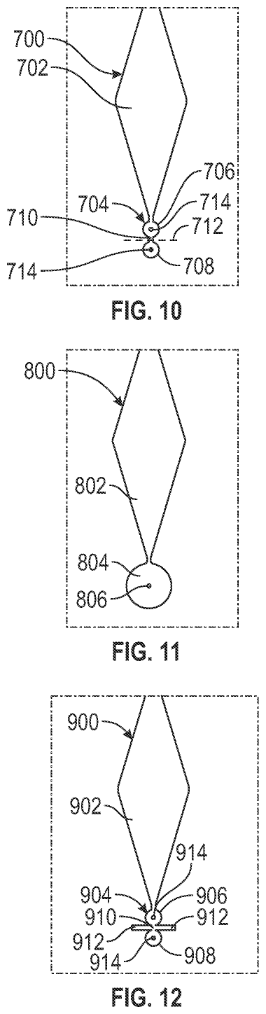

In lieu of or in addition to the openings in the apices, the protective covers can have one or more attachment features (e.g., openings, tabs, etc.) configured for coupling the protective covers to the apices of the frame. Several exemplary attachment features are described below with respect to FIGS. 10-12.

The protective covers 606 of the docking station 600 are formed as separate components from the sealing skirt 604. In some examples, one or more of the protective covers can be integrally formed with or directly coupled to the sealing skirt. For example, the protective covers disposed at the inflow end of docking station can be integrally formed with the sealing skirt, and the protective covers disposed at the outflow end of the docking station can be formed as separated components from the sealing skirt. In some examples, both the protective covers at the inflow end and the protective covers at the outflow end can be integrally formed with or directly coupled to a sealing skirt.

Referring to FIG. 8, the docking station 600 can include one or more radiopaque markers 626, which can assist with deployment of the docking station 600 as well as placement of the valve 200 into the valve seat 116. The one or more radiopaque markers 626 can be radiopaque or have a higher radiopacity one or more other components such that the one or more radiopaque markers 626 can be identified under fluoroscopy or a similar imaging process. The one or more radiopaque markers 626 can be disposed on, attached to, or otherwise affixed to the docking station 600 in a wide variety of ways, such as the ways detailed below. The one or more radiopaque markers 626 can comprise any material or combination of materials that are radiopaque or increase the radiopacity of at least a portion of the valve seat 116. For example, the one or more radiopaque markers 626 can comprise barium sulfate, bismuth, tungsten, tantalum, platinum-iridium, gold, and/or any other material which is opaque to fluoroscopy, X-rays, or similar radiation or any combination thereof. As illustrated, the radiopaque markers can be disc-shaped and circular or octagonal. However, the one or more radiopaque markers can be configured to reduce axial motion and can be any suitable shape. For example, the one or more radiopaque markers can be hexagonal, triangular, rectangular, elliptical, or any other shape or configuration. The radiopaque markers 626 can also include an aperture extending through a central portion of the marker 626. The aperture can be sized such that a suture 628 can pass therethrough. The suture 628 can also act as a belt supporting or reinforcing the valve seat 116. In this manner, the suture 628 can, for example, help ensure that the prosthetic valve is securely coupled to the docking station when the prosthetic valve is expanded within the valve seat. Additional information about the radiopaque markers and the suture can be found, for example, in International Publication No. WO 2021/188278, which is incorporated by reference herein.

Any of the docking stations disclosed herein can comprise a sealing skirt, one or more radiopaque markers, and/or a suture/belt similar to those described for the docking station 600.

FIG. 10 depicts a portion of a sealing skirt 700 comprising a main portion 702 and a protective cover portion 704. In other words, the protective cover portion 704 is integrally formed with the main portion 702. The main portion 702 of the sealing skirt 700 is configured similar to the sealing skirt 604 of the docking station 600.

The protective cover portion 704 of the sealing skirt 700 comprises a first segment 706 (e.g., an inner segment), a second segment 708 (e.g., an outer segment), and a connection segment 710 disposed between the first and second segments. In the depicted configuration, the protective cover portion 704 comprises a “figure 8” or “hourglass” type shape. In this manner, the first segment 706 of the protective cover portion 704 can be disposed on one surface (e.g., an inner surface) of an apex of the frame, and the protective cover portion 704 can be folded at the connection segment 710 (e.g., along a fold line 712) such the second segment 708 of the protective cover portion is disposed on another surface (e.g., an outer surface) of the apex of the frame.

The protective cover portion 704 also comprises a plurality of attachment openings 714. The attachment openings 714 can be configured for coupling the protective cover portion to the apex of the frame (e.g., via one or more sutures). In the illustrated example, the first segment 706 comprises a first attachment opening 714, and the second segment 708 comprises a second attachment opening 714. In some examples, each of the first and second segments can have more or less than one attachment opening formed therein.

FIG. 11 depicts a portion of a sealing skirt 800, according to one example. The portion of the sealing skirt comprises a main portion 802 and a protective cover portion 804, which are integrally formed together. The main portion 802 of the sealing skirt 800 is configured similar to the main portion 702 of the sealing skirt 700.

The protective cover portion 804 of the sealing skirt 800 comprises a single segment. The protective cover portion 804 is configured to be relatively larger than a single surface (e.g., an inner surface) of the apex of the frame. In this manner, the “oversized” protective cover can wrap around from a first surface (e.g., the inner surface) to one or more other surfaces (e.g., the side and/or outer surfaces) of the apex.

The protective cover portion 804 also comprises an attachment opening 806. The attachment opening 806 can, for example, be used to facilitate coupling to the apex of the frame (e.g., via one or more sutures). It should be noted herein the any of the “openings” disclosed herein can also be referred to as “apertures.”

FIG. 12 depicts a portion of a sealing skirt 900 comprising a main portion 902 and a protective cover portion 904, which are integrally formed together. The main portion 902 of the sealing skirt 900 is configured similar to the main portion 702 of the sealing skirt 700.

The protective cover portion 904 of the sealing skirt 900 comprises a first segment 906 (e.g., an inner segment), a second segment 908 (e.g., an outer segment), a connection segment 910 disposed between the first and second segments, and a plurality of extension segments 912 extending laterally from the connection segment 910. In the depicted configuration, the protective cover portion 904 comprises a “figure 8” or “hourglass” type shape with two arms extending outwardly therefrom. In this manner, the first segment 906 of the protective cover portion 904 can be disposed on one surface (e.g., an inner surface) of an apex of the frame, and the protective cover portion 904 can be folded at the connection segment 910 such the second segment 908 of the protective cover portion is disposed on another surface (e.g., an outer surface) of the apex of the frame. Before or after the second segment 908 is folded, the extension segments 912 can be wrapped around the apex. In this manner, the extension segments 912 can, for example, cover the side surfaces of the apex and/or help secure the protective cover portion to the apex.

The protective cover portion 904 also comprises a plurality of attachment openings 914. The attachment openings 914 can be configured for coupling the protective cover portion 904 to the apex of the frame (e.g., via one or more sutures). In the illustrated example, the first segment 906 comprises a first attachment opening 914, and the second segment 908 comprises a second attachment opening 914. In some examples, each of the first and second segments can have more or less than one attachment opening formed therein.

The portions of the sealing skirts depicted in FIGS. 10-12 are inflow end portions of sealing skirts. In some examples, the portions of the sealing skirts depicted in FIGS. 10-12 can be used on the outflow end portion of a frame.

The protective cover portions disclosed herein can comprise various sizes and/or shapes, which may or may not correspond to the shape of the apex to which the protective cover is attached. For example, in some instances, the protective cover can comprise a similar size and/or shape (e.g., circular) that corresponds to the size and shape of the apex of the frame. In some examples, the protective cover can be a different size (e.g., larger) and/or shape (e.g., rectangular) than the corresponding apex of the frame.

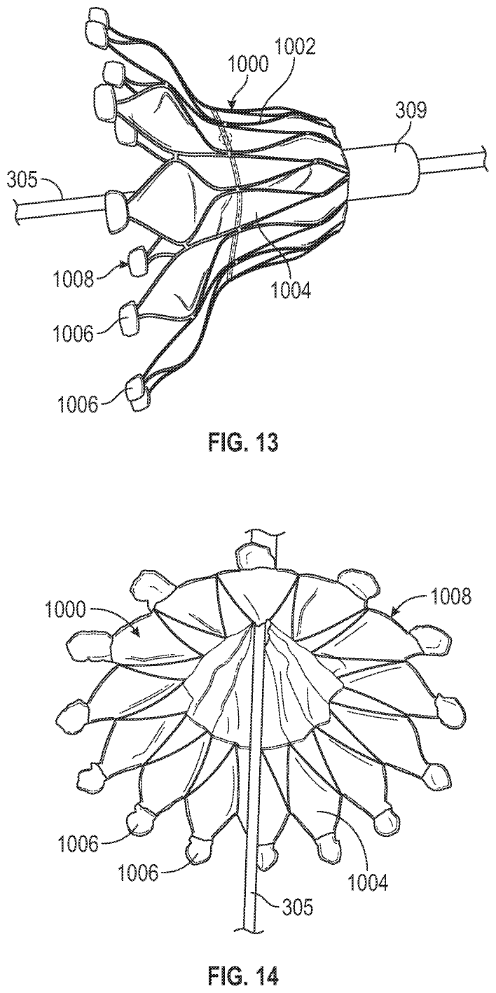

FIGS. 13-14 depict a docking station 1000, according to another example. The docking station 1000 is depicted partially deployed from the delivery apparatus 300 (e.g., similar to the configuration depicted in FIG. 6D).

The docking station 1000 comprises a frame 1002, a sealing skirt 1004, and a plurality of protective covers 1006. The frame 1002 is similar to the frame 602. The sealing skirt 1004 is similar to the sealing skirt 604, except the sealing skirt 1004 extend all the way to the apices at the outflow end 1008 of the docking station 1000. The protective covers 1006 can be configured similar to the protective covers 606.

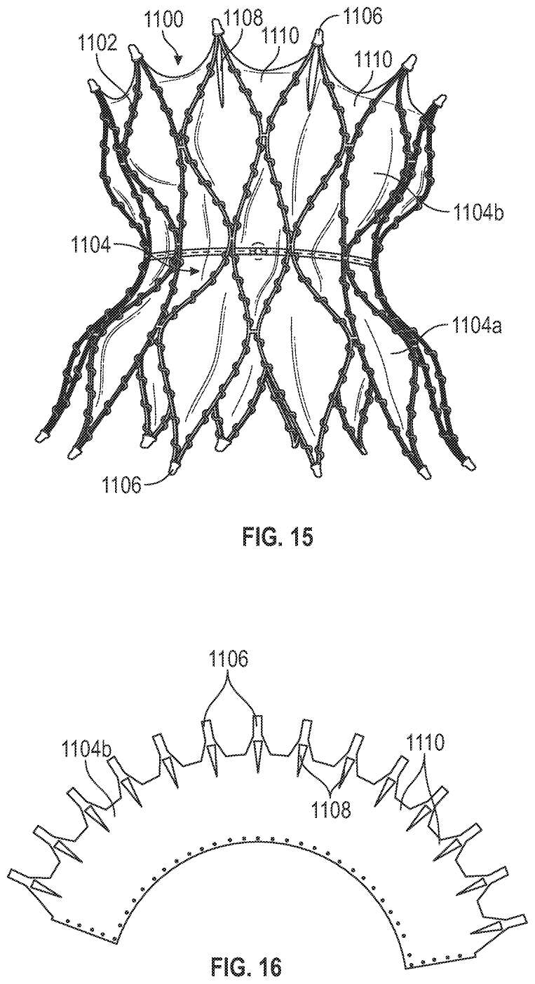

FIGS. 15-16 depict a docking station 1100 and its components, according to another example. The docking station 1000 comprises a frame 1102 and a scaling skirt 1104. The sealing skirt 1104 comprises protective cover portions 1106, which extend onto the apices of the frame 1102.

The sealing skirt 1104 is formed as a plurality of separate segments, including an inflow portion 1104a and an outflow portion 1104b. The inflow and outflow portions of the scaling skirt 1104 can be coupled together in various ways (e.g., sutures, fasteners, adhesive, and/or other means for coupling). In some examples, the inflow and outflow portions (and/or other portions of the sealing skirt) can be integrally formed as a single, unitary component. In some examples, the sealing skirt can comprise more than two separate portions (e.g., 3-5). In particular examples, the sealing skirt can comprise three separate portions, including an inflow portion, an outflow portion, and an intermediate portion disposed between the inflow and outflow portions.

In the illustrated example, the inflow portion 1104a of the sealing skirt 1104 covers all of the inflow cells of the frame 1102. In some examples, the inflow portion of the sealing skirt can cover less than all of the inflow cells of the frame 1102 (e.g., 50-99% or 75%-95%).

The outflow portion 1104b of the sealing skirt 1104 covers less than all of the outflow cells of the frame 1102 and includes a plurality of openings 1108. By covering some but not all of the outflow cells, the sealing skirt 1104 can, for example, reduce retrograde blood flow, while permitting antegrade blood flow and/or catheter access to the left and right pulmonary arterial branches.

In the depicted configuration, the outflow portion covers 75% (or approximately 75%, i.e.., 75% +/−5%) of outflow cells of the frame 1102. In some examples, the outflow portion of the sealing skirt can cover more or less of the outflow cells than depicted in illustrated example. For example, the outflow portion of the sealing skirt can cover 20-95% (or 25-85% or 70-80% in some examples) of the outflow cells of the frame 1102. In some examples, the outflow portion can cover less than 20% of the outflow cells of the frame (e.g., approximately 10%).

The openings of the sealing skirt can comprise various shapes and/or sizes. For example, the opening 1108 of the sealing skirt 1104 comprise a polygonal shape (e.g., triangular, diamond, rectangular, etc.). In some examples, the openings of the sealing skirt and comprise a rounded shape (e.g., circular, ovular, etc.). In some examples, an opening of the sealing skirt can comprise one or more rounded (or curved) edges and one or more straight edges.

In the illustrated example, the openings 1108 of the sealing skirt 1104 comprise a uniform size and shape. In some examples, the openings of the sealing skirt can comprise a non-uniform size and/or shape.

The protective cover portions 1106 (which can also be referred to as “tabs”) of the outflow portion 1104b of the sealing skirt 1104 can be configured to wrap around the outflow apices of the frame 1102. As such, the protective cover portions 1106 can, for example, reduce the potential for the apices of the frame to damage (e.g., penetrate too deeply and/or tear) the native tissue.

The protective cover portions 1106 (and/or other portions of the sealing skirt 1104) can be coupled to the frame in various ways (e.g., sutures, fasteners, adhesive, etc.).

The outflow portion 1104b of the sealing skirt 1104 also comprises extension segments 1110 configured to extend circumferentially between adjacent outflow cells of the frame 1102.

FIG. 17 depicts a portion (e.g., an outflow portion) of a sealing skirt 1200. The sealing skirt 1200 is configured similar to the outflow portion 1104b of the sealing skirt 1104. One difference between the sealing skirt 1200 and the sealing skirt 1104 is that the openings 1202 of the sealing skirt 1200 are larger than the openings 1108 of the sealing skirt 1104. Another difference between the sealing skirt 1200 and the sealing skirt 1104 is that protective cover portions 1204 of the sealing skirt 1200 comprise a disk shape and the protective cover portions 1106 of the sealing skirt 1104 comprise a rectangular shape.

The sealing skirt 1200 also comprises apertures 1206 formed in the protective cover portions 1204. The apertures 1206 can be used, for example, to couple the protective cover portions 1204 to the apices of a frame.

In some examples, each apex of the frame has a protective cover portion disposed thereon. In some examples, one or more protective cover portions can be omitted such that one or more of the apices of the frame are exposed and one or more of the apices of the frame are covered.

It should be noted that the “protective covers” and/or “protective cover portions” disclosed herein can also be referred to as “apex covers,” “pads,” and “guards.”

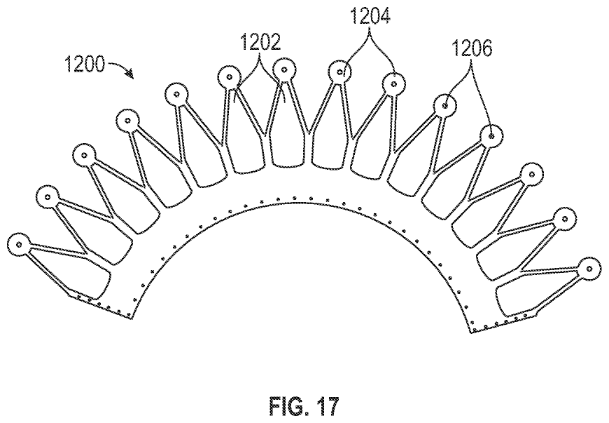

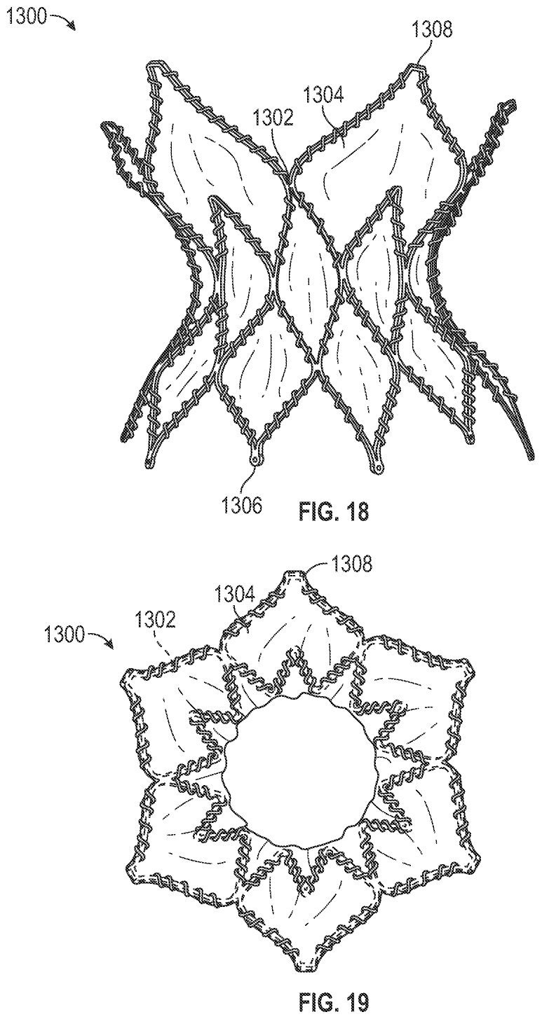

FIGS. 18-19 depict a docking station 1300, according to another example. The docking station comprises a frame 1302, a sealing skirt 1304, an inflow end 1306, and an outflow end 1308. The frame 1302 comprises relatively large cells disposed adjacent the outflow end 1308 of the docking station 1300. Forming the frame 1302 with relatively larger cells adjacent the outflow end 1308 can distribute the forces from the outflow end of the docking station on the native tissue (e.g., a native pulmonary artery) over a relatively larger area of the native tissue. This can, for example, reduce damage to the native tissue.

The frame 1302 of the docking station 1300 comprises a plurality of struts which form a plurality of cells and apices. The number of cells and apices can vary. For example, as illustrated, the frame 1302 comprises 12 inflow cells and 12 inflow apices and six outflow cells and six outflow apices. In some examples, the frame can comprise less than 12 (e.g., 9-11) or more than 12 (e.g., 12-16) inflow cells and/or inflow apices, and/or the frame can comprise less than six (e.g., 3-5) or more than six (e.g., 7-9) outflow cells and/or outflow apices.

The frame 1302 also includes a plurality of intermediate cells disposed between the inflow cells and the outflow cells. In other words, the frame 1302 comprises three circumferentially-extending rows of cells (i.e.., an inflow row, an intermediate row, and an outflow row). The frame 1302 comprises 12 intermediate cells with 12 apices directed toward the inflow end 1306 and 12 apices directed to the outflow end 1308. In some examples, the frame can comprise less or more than 12 intermediate cells and/or apices.

The docking station 1300 can, in some instances, further comprise one or more protective covers disposed on the apices (e.g., inflow and/or outflow apices) of the frame. The protective covers, together with the large outflow cells, can reduce native tissue damage, as one advantage. The protective covers and the sealing skirt 1304 can be integrally formed or formed as separate components.

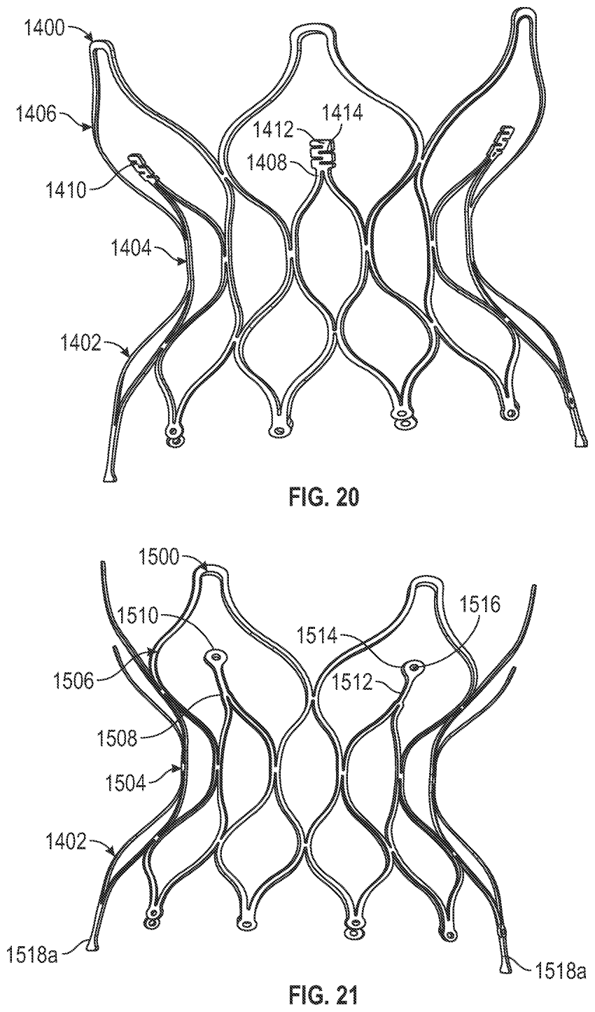

FIG. 20 depicts a frame 1400 of a docking station, according to yet another example. The frame 1400 is configured similar to the frame 1302, except the frame 1400 comprises additional force-dispersion features, which are further described below.

The frame 1400, like the frame 1302, comprises three circumferentially-extending rows of cells, including an inflow row 1402, an intermediate row 1404, and an outflow row 1406. The intermediate row 1404 of cells forms apices 1408 directed toward the outflow row 1406 of cells.

The frame 1400 comprises force-dispersion features extending from the apices 1408. In the illustrated example, the force-dispersion features comprise a plurality of flexible struts 1410, each extending from a respective apex 1408. Each flexible strut 1410 comprises a serpentine segment 1412 and one or more slots 1414. The slots extend laterally are formed in an axially-offset and alternating pattern (e.g., left side-right side-left side-right side in the depicted orientation). In this manner, the flexible struts 1410 form a spring-like pad configured to contact and conform to the native anatomy. As such, the flexible struts 1410 can, for example, help reduce the risk of native tissue damage at or proximate the apices 1408.

The frame 1400 (and/or any frame disclosed herein) can have one or more sealing skirts and/or one or more protective covers coupled thereto.

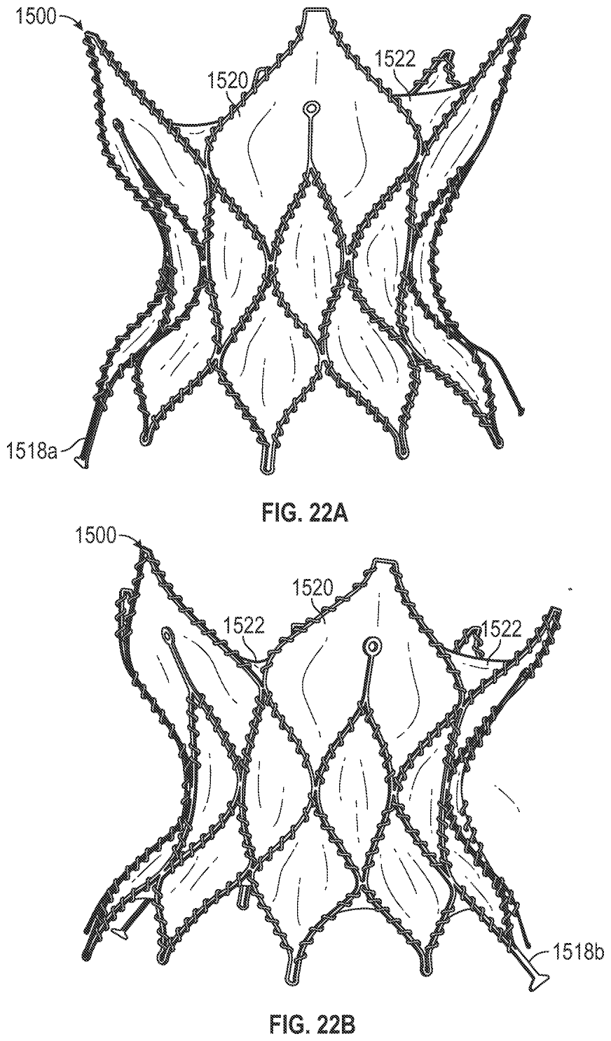

FIG. 21-22B depicts a frame 1500 of a docking station, according to yet another example. The frame 1500 is configured similar to the frame 1400, except the frame 1500 comprises an alternative example of force-dispersion features, which are further described below.

The frame 1500 comprises three circumferentially-extending rows of cells, including an inflow row 1502, an intermediate row 1504, and an outflow row 1506. The intermediate row 1504 of cells forms apices 1508 directed toward the outflow row 1506 of cells.

The frame 1500 comprises force-dispersion features extending from the apices 1508. In the illustrated example, the force-dispersion features comprise a plurality paddles 1510, each extending from a respective apex 1508. Each paddle 1510 comprises a neck portion 1512 and a head portion 1514. The neck portion 1512 comprises a first end portion coupled to and extending from an apex 1508 in a cantilevered manner. The head portion 1514 extends from a second, opposite end of the neck portion 1512. The neck portion 1512 is relatively thin and flexible (e.g., compared to the head portion 1514), which can, for example, enable the neck portion to conform to the native anatomy. The head portion 1514 is relatively large (e.g., compared to the neck portion 1512 and/or the apex 1508). As such, the head portion can, for example, distribute the contact forces over a relatively large area of the native tissue. This can, as one example, reduce damage to the native tissue at or proximate the apices 1508.

The paddles 1510 also comprise apertures 1516. The apertures 1516 can, for example, increase flexibility of the head portion of the paddles and/or provide a means for coupling a sealing skirt and/or a protective cover to the paddle.

In some examples, a frame can have force-dispersion features (e.g., the flexible struts 1410 and/or the paddles 1510) coupled to one or more apices of the frame, and other apexes of the frame can be formed without the force-dispersion features.

In some examples, a frame can comprise various combinations of force-dispersion features. For example, a frame can comprise one or more flexible struts 1410 extending from one or more apices of the frame and comprise one or more paddles 1510 extending from one or more other apices of the frame.

The frame 1500 can also comprises a plurality of connector tabs, which can be configured for releasably coupling the frame to a delivery apparatus. The connector tabs can be curved and/or angled in various ways. For example, as depicted in FIGS. 21-22A, the connector tabs 1518a are curved/angled at the same or a similar manner in which the other inflow apices of the frame 1500 are curved/angled. As another example, as depicted in FIG. 22B, the connector tabs 1518b of the frame 1500 are curved/angled differently (e.g., further outwardly) than the other inflow apices of the frame 1500 (and/or compared to the connector tabs 1518a). Configuring the connectors tabs 1518b to flare outwardly in this manner can, for example, help the frame release from the frame connector 400.

Referring to FIGS. 22A-22B, the frame 1500 can also have a scaling skirt 1520 coupled thereto, which forms a docking station. The sealing skirt 1520 is configured to cover all or at least substantially all of the outflow cells of the frame 1500. The scaling skirt 1520 also comprises extension flaps 1522 disposed between adjacent outflow cells of the frame 1500. The extension flaps can, for example, promote tissue ingrowth and/or improved sealing. The extension flaps can comprise various shapes (e.g., a convex shape, a concave shape, a straight shape, etc.). Each of the extension flaps can comprise a uniform size and shape in some examples. In some examples, one or more of the extension flaps can comprise a non-uniform shape and/or size relative to one or more other extension flaps.

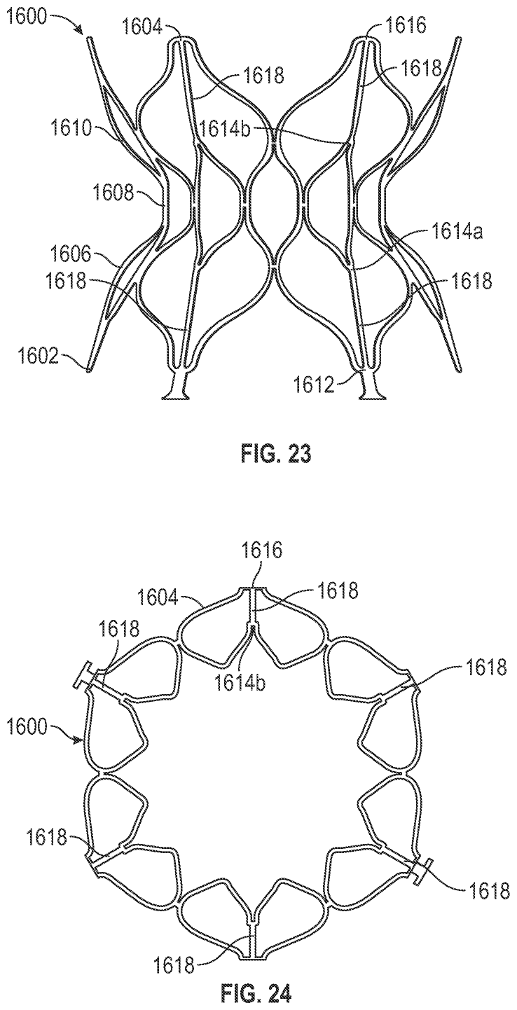

FIGS. 23-24 depict a frame 1600 of a docking station, according to another example. The frame 1600 comprises relatively large cells disposed adjacent an inflow end 1602 of the frame 1600 and relatively large cells disposed adjacent an outflow end 1604 of the frame 1600. Forming the frame 1600 with relatively larger cells adjacent the inflow and outflow ends of the frame can distribute the forces from the frame on the native tissue (e.g., a native pulmonary artery) over a relatively larger area of the native tissue. This can, for example, reduce damage to the native tissue.

The frame 1600 comprises a plurality of struts which form a plurality of cells and apices. The number of cells and apices can vary. The number of rows of cells can also vary. For example, as illustrated, the frame 1600 comprises three rows of cells: an inflow row 1606, an intermediate row 1608, and an outflow row 1610. The inflow row comprises six inflow cells and six inflow apices 1612. The intermediate row comprises 12 intermediate cells and 12 inflow-directed intermediate apices 1614a and 12 outflow-directed intermediate apices 1614b (collectively or generically referred to as “the intermediate apices 1614”). The outflow row comprises six outflow cells and six outflow apices 1616.

In some examples, a frame can comprise less or more than three rows of cells and/or each row of cells can have more or fewer cells than depicted in the illustrated examples.

The frame 1600 further comprises a plurality of support struts 1618. The support struts 1618 can, for example, increase the strength of the frame 1600. Each support strut 1618 extends from an intermediate apex 1614 to either an inflow apex 1612 or an outflow apex 1616.

The frame 1600 comprise 12 support struts 1618. In some examples, the frame can comprise more or fewer than 12 support struts. For example, in some examples, the frame can comprise three supports struts disposed at the inflow end portion and three support struts disposed at the outflow end portion. In some implementations, the inflow support struts can be circumferentially offset relative to the outflow support struts. In some implementations, the inflow support struts can be circumferentially aligned with the outflow support struts. In some examples, a frame can comprise one or more support struts at the inflow end portion of the frame and omit support struts from the outflow end portion, or vice versa.

The frame 1600 can, in some instances, further comprise one or more sealing skirts coupled to the frame and/or one or more protective covers disposed on the apices (e.g., inflow and/or outflow apices) of the frame. The protective covers, together with the large inflow/outflow cells, can reduce native tissue damage, as one advantage. The protective covers and the sealing skirt 1304 can be integrally formed or formed as separate components.

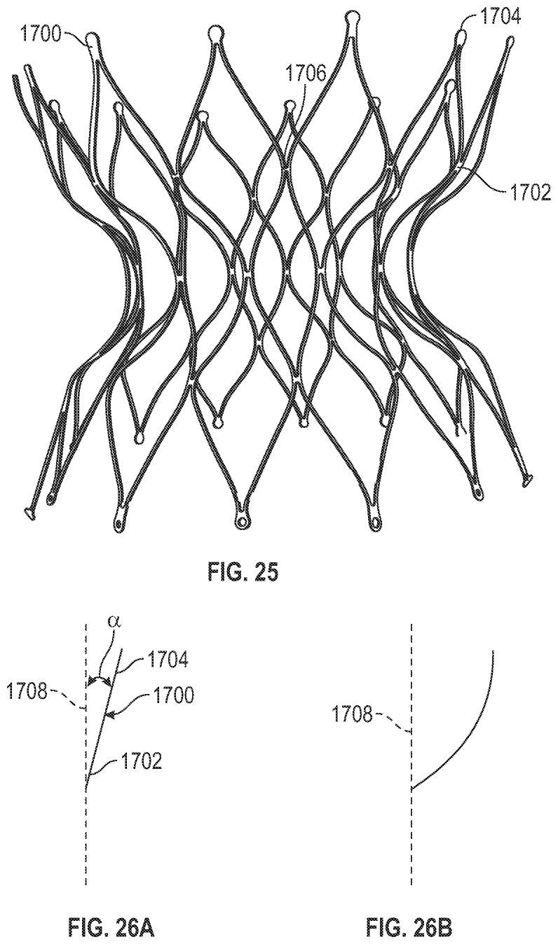

FIG. 25-26A depicts a frame 1700 of a docking station, according to another example. The frame 1700 is similar to the frame described above, except the frame has a radially tapered shape at the outflow end portion of the frame rather than a radially curved shape. The tapered shaped compared to the radially curved shape is depicted schematically in FIGS. 26A and 26B, respectively. The radially tapered shape of the outflow end portion of the frame 1700 can, for example, help ensure that a greater portion of the outflow end portion contacts the native tissue (e.g., a pulmonary artery), thereby spreading the forces more evenly across the native tissue. As such, the radially tapered shape can help reduce damage to the native tissue.

In particular, the frame 1700 comprises a radially tapered shape from a shoulder region 1702 of the frame to an outflow end 1704 of the frame. In the illustrated example, the shoulder region 1702 of the frame 1700 corresponds to the outflow directed apices 1706 of the intermediate cells of the frame, which also corresponds to the midpoint of the outflow cells of the frame. In some examples, the shoulder region can correspond to various other axial locations along the frame (e.g., either further toward the outflow end or further toward the inflow end).

Referring to FIG. 26A, the degree of the taper of the frame can vary. For example, in some examples, an angle a between the tapered region and a vertical axis 1708 can be within a range of 5-45 degrees (or 10-25 degrees or 15-20 degrees).

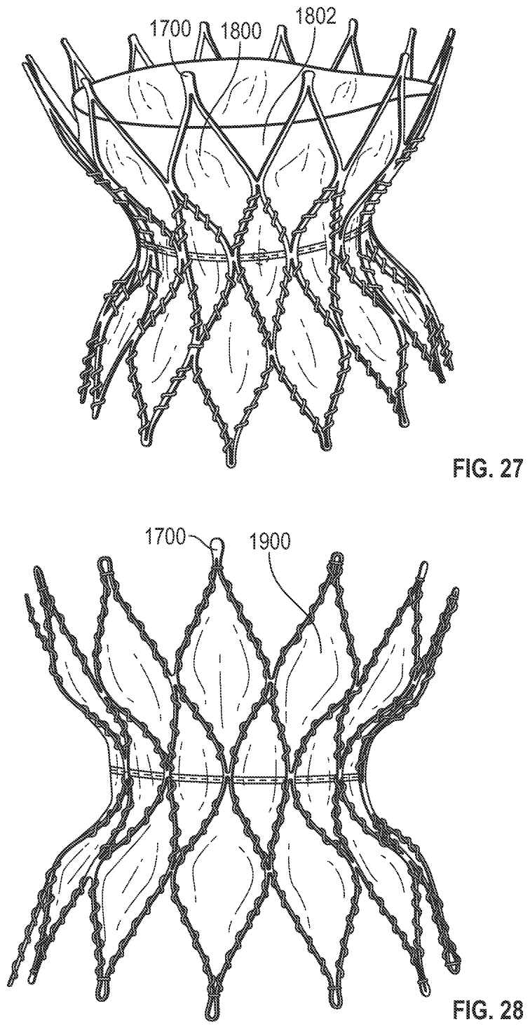

FIG. 27 depicts the frame 1700 with a sealing skirt 1800 coupled thereto. The scaling skirt 1800 is configured to cover less than all of the outflow cells of the frame. For example, in some examples, the sealing skirt 1800 can cover about 60-90% of the outflow cells of the frame. In particular examples, the sealing skirt 1800 can cover about 70-80% (or 72-77%) of the outflow cells of the frame. The sealing skirt 1800 also comprises extension regions 1802 extending between adjacent outflow cells.

FIG. 28 depicts the frame 1700 with a sealing skirt 1900 coupled thereto. The sealing skirt 1900 is configured to cover all or at least substantially all (i.e.., 95-100%) of the outflow cells of the frame 1700.

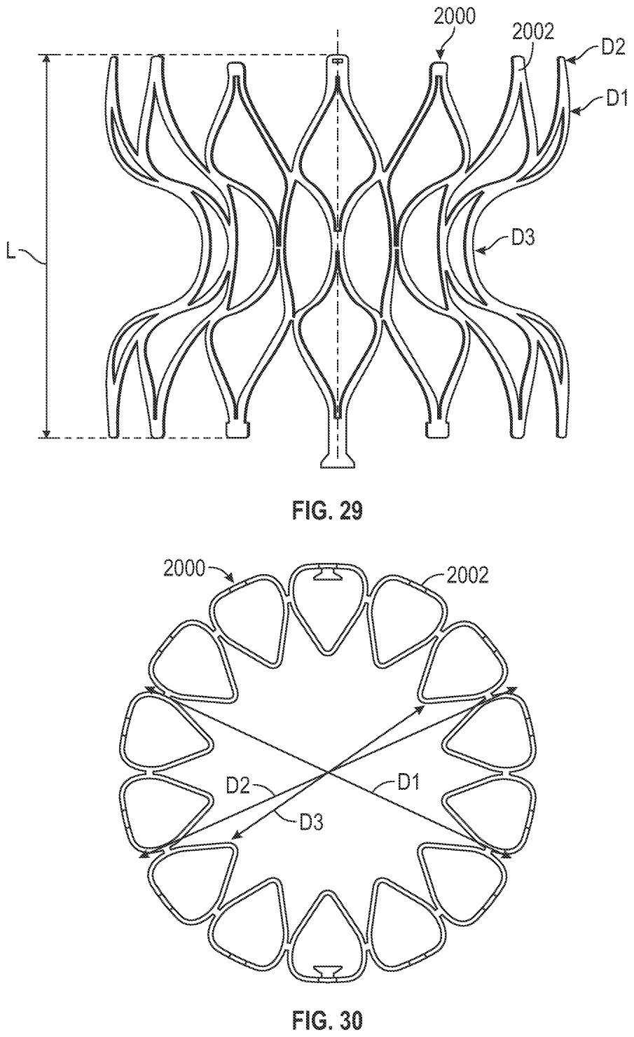

FIGS. 29-30 depict a frame 2000 of a docking station. The frame 2000 has a varying diameter along the axial length L of the frame. For example, the frame 2000 comprises a maximum diameter D1, which corresponds to the axial position of the frame depicted in FIG. 29, an outflow diameter D2, which corresponds to the outflow end of the frame, and a minimum diameter D2, which corresponds to the axial position of the frame depicted in FIG. 29. The diameter D1 >the diameter D2>the diameter D3. The diameter D2 is only slightly smaller than the diameter D2. For example, in some instances, the diameter D2 is 0-1 mm less than the diameter D1. In some instances, the diameter D2 is 0.25-0.75 mm (0.40-0.50 mm) less than the diameter D1. In some examples, the frame 2000 comprises a ratio of D2/D1 of 0.95-0.99.

As such, outflow apices 2002 are curved or angled slightly radially inwardly. This can, for example, help to prevent the outflow apices 2002 from damaging the native tissue.

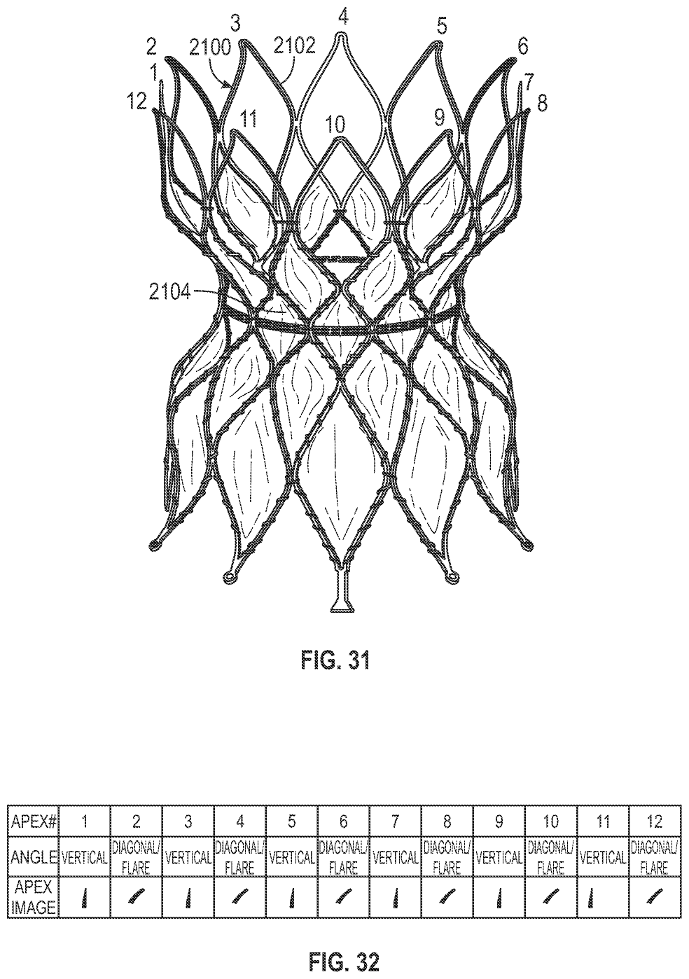

FIG. 31 depicts a docking station 2100, according to another example. The docking station 2100 comprises a frame 2102 and a sealing skirt 2104. Outflow apices (numbered 1-12) of the frame 2102 comprise various angles. For example, the odd numbered apices (i.e., 1, 3, 5, 7, 9, and 11) of the frame 2102 comprise a vertical configuration, and the even numbered apices (i.e.., 2, 4, 6, 8, 10, and 12) of the frame 2102 comprise a diagonal/flared configuration, as depicted in the table provided in FIG. 32.

The every-other type pattern depicted for the frame 2102 is exemplary. Various other patterns can be used. For example, a pattern of two vertical-two diagonal/flared can be used.

For example, in the illustrated configuration, the vertical outflow apices comprise an angle of zero degree relative to a vertical axis, and the diagonal/flared apices comprise an angle of about 45 degrees relative to a vertical axis. In some examples, the angle of the outflow apices can vary. For example, one set of apices can comprise an angle of 0-15 degrees, and another set of apices can comprise an angle of 16-55 degrees. In other words, the frame has one or more apices with a first angle and one or more other apices with a second angle.

By providing one or more apices with less angle and one or more apices with more of an angle, the frame can balance the desire to reduce tissue damage (e.g., via the less angled apices) and to also to provide adequate anti-migration relative to the native anatomy (e.g., via the more angled apices).

In some examples, the less angled apices can be oriented to a particular location in the native anatomy. For example, in some instances, one or more less angled apices can be oriented toward a medial segment of the native pulmonary artery, which is disposed adjacent the native aorta.

In lieu of or in addition to the outflow apices, the inflow apices of the frame can have varying angles.

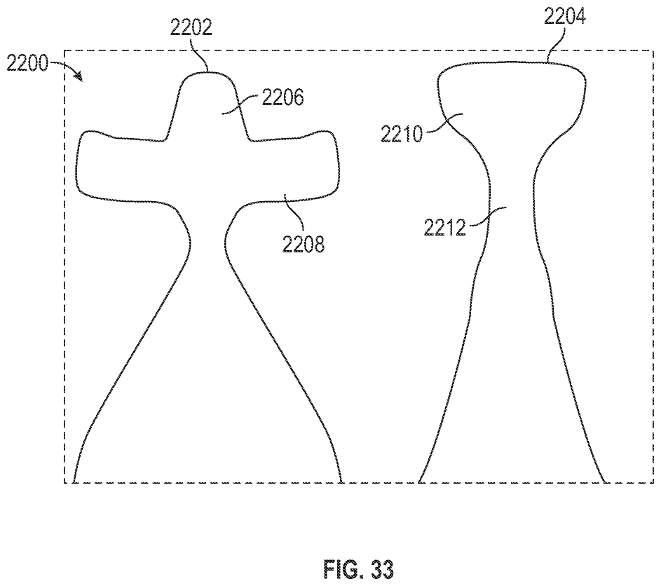

FIG. 33 depicts a portion of a frame 2200 for a docking station, which is shown schematically. The frame 2200 comprises apices (e.g., outflow apices) that have an alternating pattern (only one pair shown). A first apex 2202 of the frame 2200 has a cruciform or “t” shape, and a second apex 2204 has a tapered or rounded shape. The first apex 2202 is configured to penetrate the native tissue to a certain extent, and the second apex is configured to engage the native tissue with little or no penetration. This alternating pattern can provide adequate retention relative to the native tissue and/or reduce damage to the native tissue.

The first apex 2202 of the frame 2200 includes a projection portion 2206 and a guard portion 2208. The projection portion 2206 can be configured to penetrate the native tissue, and the guard portion 2208 can be configured to limit the extent to which the projection portion 2206 can penetrate the native tissue. As such, the guard portion 2208 acts as a stopper for the projection portion. In this manner, the first apex is similar to a knife where the projection portion 2206 is like the blade of the knife and the guard portion 2208 is like the quillon of the knife.

The second apex 2204 of the frame 2200 includes a tapered shape having a wide portion 2210 and a narrow portion 2212. In some examples, the wide portion 2210 of the second apex 2204 can be axially aligned with the projection portion 2206 of the first apex 2202, and the narrow portion 2212 of the second apex 2204 can be axially aligned with the guard portion 2208 of the first apex 2202. Configuring the first apex 2202 and the second apex 2204 in this manner can for example a relatively wide portion of the first apex 2202 to nest with a relatively narrow portion of the second apex 2204, and vice versa. This can, for example, reduce the diameter to which the frame 2200 can be radially compressed and/or prevent the apices from contacting each other when radially compressed.

In some examples, a frame can comprise a plurality of apices configured similar to the first apex 2202 that are disposed adjacent each other. In such examples, the guard portions of the first apices can be axially offset relative to each other to prevent the guard portions from contacting each other when the frame is radially compressed.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A docking station for a prosthetic implant, including a frame and one or more protective covers. The frame includes a plurality of struts, and the struts form one or more apices. The protective covers are disposed on the apices and are configured to be positioned between the apices of the frame and native tissue at an implantation location.

Example 2. The docking station of any example herein, and particularly example 1, wherein the one or more protective covers comprise PET, PTFE, ePTFE, or TPU.

Example 3. The docking station of any example herein, and particularly either example 1 or example 2, further comprising one or more sealing skirts coupled to the plurality of struts of the frame.

Example 4. The docking station of any example herein, and particularly example 3, wherein the one or more protective covers are integrally formed with the sealing skirt.

Example 5. The docking station of any example herein, and particularly example 3, wherein the one or more protective covers are formed as separate components from the sealing skirt.

Example 6. The docking station of any example herein, and particularly example 3, wherein at least one of the one or more protective covers is integrally formed with the sealing skirt.

Example 7. The docking station of any example herein, and particularly either example 3 or example 6, wherein at least one the one or more protective covers is formed as separate components from the sealing skirt.

Example 8. The docking station of any example herein, and particularly any one of examples 1-7, wherein the one or more protective covers are coupled to the plurality of struts of the frame via one or more sutures.

Example 9. The docking station of any example herein, and particularly any one of examples 1-8, wherein the one or more apices includes a first plurality of apices disposed at an inflow end of the frame and a second plurality of apices disposed at the outflow end of the frame.

Example 10. The docking station of any example herein, and particularly example 9, wherein the plurality of struts forms a first plurality of cells adjacent the inflow end of the frame and a second plurality of cells adjacent the outflow end of the frame.

Example 11. The docking station of any example herein, and particularly example 10, wherein the one or more sealing skirts completely cover the first plurality of cells.

Example 12. The docking station of any example herein, and particularly example 11, wherein the one or more sealing skirts completely cover the second plurality of cells.

Example 13. The docking station of any example herein, and particularly either example 10 or example 11, wherein the one or more sealing skirts cover less than all of each cell of the second plurality of cells.

Example 14. The docking station of any example herein, and particularly example 13, wherein the one or more sealing skirts comprise one or more openings formed therein.

Example 15. The docking station of any example herein, and particularly any one of examples 1-14, wherein the one or more protective covers comprise opening formed therein configured for coupling the one or more protective covers to the frame.

Example 16. A frame for supporting a prosthetic implant, including a first plurality of cells and a second plurality of cells. The first plurality of cells is arranged in a first circumferentially-extending row. The second plurality of cells is arranged in a second circumferentially-extending row, and the cells of the second plurality of cells are larger than the cells of the first plurality of cells.

Example 17. The frame of any example herein, and particularly example 16, wherein the first plurality of cells is disposed adjacent an inflow end of the frame.

Example 18. The frame of any example herein, and particularly either example 16 or example 17, wherein the second plurality of cells is disposed adjacent an outflow end of the frame.

Example 19. The frame of any example herein, and particularly any one of examples 16-18, further comprising a third plurality of cells disposed between the first plurality of cells and the second plurality of cells, wherein the second plurality of cells is larger than the third plurality of cells.

Example 20. The frame of any example herein, and particularly example 19, wherein the cells of the first plurality of cells are larger than the cells of the third plurality of cells.

Example 21. The frame of any example herein, and particularly example 16, further comprising a third plurality of cells, wherein the first plurality of cells is disposed between the second plurality of cells and the third plurality of cells, wherein the second plurality of cells is disposed adjacent an outflow end of the frame, wherein the third plurality of cells is disposed adjacent an inflow end of the frame, and wherein the cells of the third plurality of cells are larger than the first plurality of cells.

Example 22. The frame of any example herein, and particularly example 21, wherein the cells of the second plurality of cells are larger than the cells of the third plurality of cells.

Example 23. The frame of any one of claims 16-22, wherein the first plurality of cells comprises 10-16 cells.

Example 24. The frame of any example herein, and particularly example 23, wherein the first plurality of cells comprises 12-14 cells.

Example 25. The frame of any example herein, and particularly example 24, wherein the first plurality of cells comprises exactly 12 cells.

Example 26. The frame of any example herein, and particularly example 24, wherein the first plurality of cells comprises exactly 14 cells.

Example 27. The frame of any example herein, and particularly any one of examples 16-22, wherein the second plurality of cells comprises 4-8 cells.

Example 28. The frame of any example herein, and particularly example 27, wherein the second plurality of cells comprises 5-6 cells.

Example 29. The frame of any example herein, and particularly example 28, wherein the second plurality of cells comprises exactly six cells.

Example 30. The frame of any example herein, and particularly any one of examples 1-29, wherein the frame comprises one or more force-dispersion features.

Example 31. The frame of any example herein, and particularly example 31, wherein the one or more force-dispersion features comprises one or more flexible struts coupled to the plurality of struts, each flexible strut comprising a serpentine portion and a plurality of notches.

Example 32. The frame of any example herein, and particularly either example 31 or example 32, wherein the one or more force-dispersion features comprises one or more paddles coupled to the plurality of struts, each paddle comprising a neck portion and a head portion.

Example 33. The frame of any example herein, and particularly any one of examples 1-32, further comprising one or more connector tabs configured for coupling the frame to a delivery apparatus.

Example 34. The frame of any example herein, and particularly example 33, wherein the connector tabs follow a curvature of the frame at an inflow end of the frame.

Example 35. The frame of any example herein, and particularly example 33, wherein the connector tabs flare radially outwardly relative to apices at an inflow end of the frame.

Example 36. A sealing skirt configured to be coupled to any frame herein, and particularly any one of the frames of examples 1-35, comprising a first portion and a second portion. The first portion is configured to cover one or more cells of the frame, and the second portion is configured to extend between adjacent cells of the frame.

Example 37. A frame for a docking station, comprising a plurality of cells and one or more support struts. The plurality of cells is defined by a plurality of struts, and the cells comprise a first row of apices and a second row of apices. Each support strut extends axially from an apex in the first row of apices to an apex in the second row of apices.

Example 38. A frame for a docking station, comprising a plurality of struts forming a plurality of cells. The cells extend from an inflow end of the frame to an outflow end of the frame. One or more cells disposed adjacent the outflow end comprise a radially tapered section, and one or more cells disposed adjacent the inflow end comprise a radially curved section.

Example 39. A frame for a docking station, comprising an inflow end portion, an outflow end portion, and an intermediate portion. The outflow end portion has a first diameter at a first axial location and a second diameter at a second axial location. The second diameter is smaller than the first diameter. The second axial location is disposed closer to a distal end of the frame than the first axial location. The intermediate portion is disposed between the inflow end portion and the outflow end portion and having a third diameter at a third axial location. The third diameter is smaller than the first diameter and the second diameter. The third axial location is disposed closer toward an inflow end of the frame than the first axial location and the second axial location.

Example 40. The frame of any example herein, and particularly any one of examples 1-39, wherein one or more apices of the frame comprise a first configuration, and wherein one or more other apices of the frame comprise a second configuration.

Example 41. The frame of any example herein, and particularly example 40, wherein the first configuration is a vertical configuration, and wherein the second configuration is a diagonal or flared configuration.

Example 42. The frame of any example herein, and particularly either example 40 or example 41, wherein apices of the frame are arranged in an alternating pattern between the first configuration and the second configuration.

Example 43. The frame of any example herein, and particularly example 42, wherein the alternating pattern is the first configuration-the second configuration-the first configuration and so forth.

Example 44. The frame of any example herein, and particularly any one of examples 40-43, wherein the one or more apices and the one or more other apices are disposed at an outflow end portion of the frame.

Example 45. The frame of any example herein, and particularly any one of examples 1-44, wherein one or more of the apices comprises a cruciform shape.

Example 46. The frame of any example herein, and particularly example 45, wherein the one or more apices comprise a projection portion and a guard portion which form the cruciform shape.

Example 47. The frame of any example herein, and particularly any one of examples 1-46, wherein one or more of the apices comprise a tapered shape having a narrow portion and a wide portion.

Example 48. The frame of any example herein, and particularly example 47, wherein the narrow portion of the tapered shape is axially aligned with the guard portion of the cruciform shape, and wherein the wide portion of the tapered shape is axially aligned with the projection portion of the cruciform shape.

Example 49. The frame of any example herein, and particularly any one of examples 46-48, wherein the cruciform shape and the tapered shape are configured to nest with each other when the frame is radially compressed.

Example 50. A method comprising sterilizing any one of the docking stations or frames of any example herein, and particularly any one of examples 1-49.

Example 51. A method of implanting a prosthetic device comprising any one of the devices disclosed herein, and particularly any one of the devices of examples 1-49.

Example 52. A method of simulating an implantation procedure for a prosthetic device comprising any one of the devices disclosed herein, and particularly any one of the devices of examples 1-49.

The features described herein with regard to any example can be combined with other features described in any one or more of the other examples, unless otherwise stated.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

1. A docking station for a prosthetic implant, comprising:
a frame comprising a plurality of struts, wherein the plurality of struts forms one or more apices; and
one or more protective covers disposed on the apices, wherein the one or more protective covers are configured to be positioned between the apices of the frame and native tissue at an implantation location.

2. The docking station of claim 1, wherein the one or more protective covers comprise PET, PTFE, ePTFE, or TPU.

3. The docking station of claim 1, further comprising one or more sealing skirts coupled to the plurality of struts of the frame.

4. The docking station of claim 3, wherein the one or more protective covers are integrally formed with the sealing skirt.

5. The docking station of claim 3, wherein the one or more protective covers are formed as separate components from the sealing skirt.

6. The docking station of claim 3, wherein at least one of the one or more protective covers is integrally formed with the sealing skirt.

7. The docking station of claim 3, wherein at least one the one or more protective covers is formed as separate components from the sealing skirt.

8. The docking station of claim 1, wherein the one or more protective covers are coupled to the plurality of struts of the frame via one or more sutures.

9. The docking station of claim 1, wherein the one or more apices includes a first plurality of apices disposed at an inflow end of the frame and a second plurality of apices disposed at the outflow end of the frame.

10. The docking station of claim 9, wherein the plurality of struts forms a first plurality of cells adjacent the inflow end of the frame and a second plurality of cells adjacent the outflow end of the frame.

11. The docking station of claim 10, wherein the one or more sealing skirts completely cover the first plurality of cells.

12. The docking station of claim 11, wherein the one or more sealing skirts completely cover the second plurality of cells.

13. The docking station of claim 10, wherein the one or more sealing skirts cover less than all of each cell of the second plurality of cells.

14. The docking station of claim 13, wherein the one or more sealing skirts comprise one or more openings formed therein.

15. The docking station of claim 1, wherein the one or more protective covers comprise opening formed therein configured for coupling the one or more protective covers to the frame.

16. A frame for supporting a prosthetic implant, comprising:
a first plurality of cells arranged in a first circumferentially-extending row; and
a second plurality of cells arranged in a second circumferentially-extending row, wherein the cells of the second plurality of cells are larger than the cells of the first plurality of cells.

17. The frame of claim 16, wherein the first plurality of cells is disposed adjacent an inflow end of the frame.

18. The frame of either claim 16, wherein the second plurality of cells is disposed adjacent an outflow end of the frame.

19. The frame of claim 16, further comprising a third plurality of cells disposed between the first plurality of cells and the second plurality of cells, wherein the second plurality of cells is larger than the third plurality of cells.

20. The frame of claim 19, wherein the cells of the first plurality of cells are larger than the cells of the third plurality of cells.
